(19) United States
(12) Patent Application Publication
(10) Pub. No.: US 2019/0167711 A1
(43) Pub. Date: Jun. 6, 2019

(54) METHODS FOR DIAGNOSIS AND TREATMENT OF ACUTE LYMPHOBLASTIC LEUKEMIA

(71) Applicant: MOR RESEARCH APPLICATIONS LTD., Tel Aviv (IL)

(72) Inventors: Smadar AVIGAD, Petah Tikva (IL); Isaac YANIV, Petah Tikva (IL); Keren SHICHRUR, Kiryat Ono (IL)

(21) Appl. No.: 16/272,352

(22) Filed: Feb. 11, 2019

Related U.S. Application Data
(63) Continuation-in-part of application No. 15/500,456, filed on Jan. 30, 2017, now abandoned.
(60) Provisional application No. 62/030,629, filed on Jul. 30, 2014.

(51) Int. Cl. A61K 31/7105; C12Q 1/6886; A61P 35/02

(52) U.S. Cl. CPC A61K 31/7105; C12Q 2600/106; A61P 35/02; C12Q 1/6886

(57) ABSTRACT

Provided herein is the use of miR-451 as a biomarker in prognosis of anti-ALL treatment modalities, in early diagnosis of ALL relapse risk, and/or in identifying an ALL patient that can benefit from a treatment modality that affects one or more miR-451-related metabolic pathways.

### CROSS-REFERENCE TO RELATED APPLICATIONS

The present application is a continuation in part of application Ser. No. 15/500,456, filed Jul. 30, 2015, which claims benefit of provisional application No. 62/030,629, filed Jul. 30, 2014, the entire contents of both of which being hereby incorporated herein by reference.

The Sequence Listing in ASCII text file format of 1,170 bytes in size, created on Feb. 11, 2019, with the file name “2019-02-11Sequence_Listing-AVIGAD1A,” filed in the U.S. Patent and Trademark Office on even date herewith, is hereby incorporated herein by reference.

### FIELD AND BACKGROUND

The present disclosure relates to the use of micro-RNAs as biomarkers in diagnosis and treatment of acute lymphoblastic leukemia (ALL) patients.

### BACKGROUND

Leukemia is a cancer of the blood or bone marrow characterized by an abnormal increase of blood cells, usually leukocytes. Leukemia is clinically and pathologically subdivided into a variety of large groups, including its acute and chronic forms. Acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children, of which, acute lymphoblastic leukemia (ALL) is the most prevalent.

Current treatments for ALL in children are guided by patient assessment and classification into a particular risk group. Examples of such classifications include the Berlin-Frankfurt-Münster (BFM), the Children Oncology Group (COG) (Schrappe, Ann Hematol. (2004); 83: S121-S123; Vrooman L M et al., Curr Opin Pediatr. (2009); 21(1): 1-8), UKALL, from the United Kingdom, the Chinese Children's Leukemia Group (CCLG), and the Dana-Farber Cancer Institute ALL Consortium (DFCI). In the classifications, patients are classified, inter alia, based on white blood cell count, chromosomal rearrangement, and responsiveness to prednisone treatment at day 8 following treatment initiation. Classification into a particular group will determine how aggressively a patient is treated in order to provide effective treatment and to reduce the possibility of disease relapse.

While current methods of diagnosis and treatment have improved the cure rate up to 80-90%, certain children are still over- or under-treated (Schrappe M et al., Leukemia. (2010); 24: 253-254; Pui C H and Evans W E, N. Engl. J. Med. (2006); 354: 166-178; Bhojwani D et al., Clin. Lymphoma. Myeloma. (2009); 3: S222-230), mostly due to poor prognosis. Thus, a continuing need exists for improved ALL prognosis and treatment.

### SUMMARY

Improved understanding of ALL biology is necessary for the development of novel treatment strategies. A major challenge relevant to treatment modalities is to enhance the detection of those patients who eventually relapse by use of specific drug responsiveness classification. Metabolic components and metabolic pathways are becoming popular targets in cancer treatment. For example, nicotinamide phosphoribosyltransferase (NAMPT) regulates an essential metabolic pathway which is associated with cell aggressiveness that may lead to relapse in ALL patients. The present disclosure identifies NAMPT as a therapy target.

MicroRNA (miRNA) profiling identifies cancer-specific and prognostic signatures in many pediatric malignancies, including ALL. Provided herein is an ALL miRNA panel predicting relapse at the time of diagnosis without the need to wait for initial treatment response, thereby enabling early, personalized (patient-tailored) and effective therapeutic intervention. For example, the present disclosure provides miR-451 as a potential biomarker in the selection of a sub-group of ALL patients who are most likely to benefit from treatment with a NAMPT inhibitor.

Some embodiments of the present disclosure relate to the use of miR-451 as a main, or even a sole, biomarker in prognosis of anti-ALL treatment modalities, in early diagnosis of ALL relapse risk, and in identifying an ALL patient that can benefit from a treatment modality that affects one or more miR-451-related metabolic pathways, particularly when the metabolic pathway associated with miR-451 expression is NAMPT-regulated intracellular nicotinamide adenine dinucleotide (NAD) biosynthesis.

Based on determining a level of expression of miR-451 in a biological sample obtained form a subject, for example, upon first diagnosing the subject as being inflicted with ALL, a correlation of the level of expression of miR-451 with positive prognosis of a treatment modality that inhibits NAMPT and/or a relapse risk may be determined if the level of expression of miR-451 is below a predetermined level.

For example, described herein is a method for prognosis of a treatment modality that inhibits NAMPT in a subject afflicted with ALL, the method comprising:

determining a level of expression of miR-451 in a biological sample obtained from the patient; and if the level of expression of miR-451 is below a predetermined level, correlating the level of expression of miR-451 with a positive prognosis of a treatment modality that inhibits NAMPT.

Also, described herein is a method for identifying ALL patients most likely to benefit from treatment with a NAMPT inhibitor, comprising:

obtaining a biological sample from an ALL patient;

determining a level of expression of miR-451 in the biological sample; and if the level of expression of miR-451 is below a predetermined level, identifying the patient as being likely to benefit from treatment with a NAMPT inhibitor.

Further, disclosed herein is method for early diagnosis of ALL relapse risk in a subject, comprising:

obtaining a biological sample from the subject when first diagnosed as inflicted with ALL;

detecting a level of expression of miR-451; and diagnosing ALL relapse risk if the level of expression of miR-451 is below a predetermined level.

Further described herein is a method for treatment of ALL in a patient that can benefit from a treatment modality that affects a metabolic pathway associated with miR-451 expression comprising:

detecting a level of expression of miR-451 in a biological sample from the patient;

correlating the level of expression of miR-451 with a positive response of the patient to a treatment modality that affects a metabolic pathway associated with miR-451 expression if the level of expression of miR-451 is below a predetermined level; and providing to the patient a treatment modality that affects a metabolic pathway associated with miR-451 expression. In exemplary embodiments, the metabolic pathway associated with miR-451 expression is NAMPT-regulated NAD biosynthesis. A non-limiting example of a treatment modality that affects this metabolic pathway is inhibition of NAMPT, for example by one or more inhibitors selected from the group consisting of a small molecule inhibitor, antibody, antisense nucleic acid, micro-RNA (miRNA) and RNA interference agent.

The novel use of miR-451 as a biomarker as provided by the present disclosure, may affords integration of miR-451 into diagnostic testing, thereby leading to potential novel therapies.

The present disclosure further relates to compositions for use in treatment of ALL. In particular embodiments, the compositions include an inhibitor of NAMPT, an inhibitor of miR-1290, a ribonucleic acid sequence at least 90% identical to a miR-451 ribonucleic acid sequence set forth as SEQ ID NO: 2, and/or an inhibitor of Janus kinase 2 (JAK2), for use in treatment of ALL.

The described compositions can all be used in methods of treatment of ALL in a subject, including reducing the risk of relapse in a subject or even preventing relapse in the subject, wherein the described compositions are administered to the subject, thereby treating ALL.

The present disclosure yet further relates to methods for treatment of ALL, wherein these methods include first determining the expression level of miR-1290 and at least one of miR-151-5p and miR-451; and comparing the determined expression of miR-1290, and miR-151-5p and/or miR-451 with control expression of miR-1290, and miR-151-5p and/or miR-451, wherein a significant increase in miR-1290 expression in the subject in comparison to the control miR-1290 expression, combined with a significant decrease in expression of the at least one of miR-151-5p and miR-451 in comparison to the control expression of miR-151-5p and/or miR-451, indicates that the subject has an increased risk of ALL relapse, and requires treatment appropriate for a subject with an increased risk of ALL relapse; and then administering to the patient a therapeutically effective amount of a composition comprising an inhibitor of NAMPT, or any of the other compounds or compositions described herein for use in treating ALL.

Further described herein is a method for treatment of pediatric ALL in a subject, comprising: administering to the subject a therapeutically effective amount of an inhibitor of NAMPT, selected from the group consisting of an antibody, antisense nucleic acid, microRNA (miRNA) and RNA interference agent, thereby treating the patient, with the proviso that the inhibitor of NAMPT is not miR-451.

In some embodiments, the ALL being treated and/or diagnosed in any of the methods disclosed herein is pediatric B-cell ALL and the patient is an adolescent, child, or infant.

The foregoing and other objects, features, and advantages will become more apparent from the following detailed description, which proceeds with reference to the accompanying figures.

### BRIEF DESCRIPTION OF THE DRAWINGS

FIG. 1 is a Kaplan Meier estimation of relapse-free survival (RFS) in a cohort of 125 acute lymphoblastic leukemia (ALL) patients. In the plot, the line representing high or low expression of miR-1290 is accordingly indicated. “n” is the number of patients assessed; “R” is the number of patients with recurrence.

FIG. 2 is a Kaplan Meier plot of relapse-free survival only for B-lineage ALL cohort (n=105). In the plot, the line representing high or low expression of miR-1290 is accordingly indicated. “n” is the number of patients assessed; “R” is the number of patients with recurrence.

FIG. 3 is a Kaplan Meier analysis for relapse-free survival by expression levels of combined miRNAs: both downregulated miRNAs (miR-451 and miR-151-5p) together with the upregulated miR-1290, in precursor B-cell ALL patients. The lower line represents a combination of down-regulated miR-451 and miR-151-5p, and up-regulated miR-1290. The upper line represents all other expression combinations for miR-451, miR-151-5p, and miR-1290. “n” is the number of patients assessed; “R” is the number of patients with recurrence.

FIGS. 4A-4C show the effect of miR-451 mimic transfection on ALL cell growth in vivo. FIG. 4A: expression analysis of miR-451 mimic measured by quantitative reverse transcription-PCR (RT-qPCR). Expression of hsa-miR-451 in Nalm-6 cells transfected with miR-451 or scrambled nucleic acid sequence (“scrambled miRNA” or “scrambled miR”) 24 hr, 5 day and 10 days after transfection. FIG. 4B: comparison of tumor size in female NOD/SCID mice transplanted with Nalm-6 cells transfected with miR-451 mimic or Nalm-6 cells transfected with scrambled-miRNA, for 31 days following sub-cutaneous (s.c.) injection of transfected cells. FIG. 4C: mean tumor weight in NOD/SCID mice transplanted with Nalm-6 cells transfected with miR-451 mimic or Nalm-6 cells transfected with scrambled-miR as measured at the end of the experiment. Vertical bars represent the standard error (SE). * denotes p<0.05.

FIGS. 5A-5B show the effect of miR-451 on NAMPT expression. FIG. 5A: expression analysis of NAMPT, as measured by FACS, in NALM-6 cell line expressing miR-451 mimic, miR-451 inhibitor (miArrest™ miR-451) or scrambled miR (control). FIG. 5B: Luciferase reporter assay validating the direct interaction of miR-451 with the 3′-UTR of NAMPT. Vertical bars represent the standard error (SE). * denotes p<0.05.

FIGS. 6A-6B show the effect of 12-O-tetradecanoylphorbol-13-acetate (TPA) on NAMPT expression and on NAD⁺ levels. FIG. 6A: expression analysis of NAMPT measured by RT-qPCR in NALM-6 cell line treated with 50 ng/ml TPA for 24 hours. FIG. 6B: NAD⁺ assay results in cells treated with 50 nM TPA for 24 hours. “NAD” represents NAD⁺/NADH ratio as measured from whole-cell extracts at 450 nm. Vertical bars represent the SE.

FIGS. 7A-7C show the effect of the NAMPT inhibitor FK866 on apoptosis, cell viability and NAD⁺ levels in NALM-6 cells. FIG. 7A: NAD⁺ levels in cells treated with FK866 for 1, 3 and 6 hours. “NAD” represents NAD⁺/NADH ratio as measured from whole-cell extracts at optical density (OD) 450 nm. FIG. 7B: apoptosis percentages in cells treated with FK866. FIG. 7C: viability of cells treated with FK866, as measured in a tetrazolium dye (XTT) viability assay. The amount of light absorbance at 450-500 nm is indicated as “cell viability”. Vertical bars represent SE. * denotes p<0.05.

FIGS. 8A-8B show the effect, in an exemplary embodiment, of the NAMPT inhibitor FK866 on NAD⁺ levels in ALL cell line transfected with miR-451 mimic (FIG. 8A), miR-451 inhibitor (FIG. 8B) or with scrambled-miRNA as negative control. Cells were treated for 3 hours with FK866. “NAD” represents NAD⁺/NADH ratio as measured from whole-cell extracts at OD 450 nm.

FIG. 9 shows SOCS4 protein expression levels following over-expression of miR-1290 in ALL cell line transfected with miR-1290 mimic in comparison to control (cells transfected with scrambled miRNA). Protein expression percentage was determined and quantified by Western blotting.

FIGS. 10A-10B show SOCS4 protein levels in ALL bone marrow (BM) samples with high or low miR-1290 levels. FIG. 10 A: representative Western blots using glyceraldehyde-3-phosphate dehydrogenase (GAPDH) levels as reference. FIG. 10B: quantitation of SOCS4 protein levels, calculated using the averaged quantification of band intensity in each group (intensity of the protein band was measured using an in-house program), and dividing each value obtained by the value of GAPDH from the same sample. The normalized ratio indicated the protein expression level in the sample. * denotes p<0.05.

FIG. 11 shows the fold change in quantity of phosphorylated-STAT protein levels following overexpression of miR-1290 in ALL cell line transfected with miR-1290 mimic in comparison to control (cells transfected with scrambled miRNA).

FIG. 12 shows miRNA-451 expression level in BM samples obtains from relapsed (n=35) and non-relapsed (n=103) ALL patients, as determined by RT-qPCR. Vertical bars represent SE. * denotes p<0.001.

FIGS. 13A-13B show relative tumor growth and tumor volume in xenograft ALL model of NSG™ mice injected with NALM-6 cells harboring antagomiR-451 (miR-451 inhibitor; n=21) or miR-451 mimic (n=22). Tumor growth was calculated as relative tumor growth compared to tumor size at day 0. FIG. 13A: relative tumor growth measured starting on the day tumors were clinically evident in all mice (day0, about 15-25 days after NALM-6 cells injection). FIG. 13B: tumor volume measured starting on day 19 (d19) after injection. Vertical bars represent SE. * denotes p<0.001.

FIG. 14 shows Western blots of NAMPT, ADAM10 and CXCL16 proteins (denoted “Targets” in the figure) in NALM-6 cell line transfected with miR-451 mimic or scrambled-miR (control). GAPDH levels serve as reference.

FIGS. 15A-15B show the effect of the NAMPT inhibitor FK866 on tumor growth in an ALL xenograft mouse model. FIG. 15A: average tumor volume measured daily in mice treated daily with 15 mg/kg of FK866 (n=41) or with saline (control; n=41). FIG. 15B: average tumor volume measured on day 32 (end of treatment) vs. day 25 (start of treatment) in FK866 treated mice and non-treated mice (control). Vertical bars represent SE. * denotes p<0.05.

FIGS. 16A-16B show the effect of miR-451 expression level on sensitivity of treatment with the NAMPT inhibitor FK866 in xenograft ALL mice model. FK866 treatment started on day 17 (d17) after induction of ALL in the mice. FIG. 16A: tumor volume measured daily in NALM-6/miR-451 mimic (red lines n=14) or NALM-6/antagomiR-451 (blue lines n=16) transduced mice treated daily with 15 mg/kg of FK866 (broken red line, n=4; and broken blue line, n=6, respectively) or saline (control) (continuous line, n=10, and continuous blue line; n=10, respectively). FIG. 16B: average tumor volume at day 30 (end of treatment) in FK866 treated mice and non-treated mice (control). Vertical bars represent SD. * denotes p<0.05; ** denotes p<0.01.

### BRIEF DESCRIPTION OF THE DESCRIBED SEQUENCES

The nucleic acid sequences provided herewith are shown using standard letter abbreviations for nucleotide bases as defined in 37 C.F.R. 1.822. Only one strand of each nucleic acid sequence is shown, but the complementary strand is understood as included by any reference to the displayed strand. In the accompanying sequence listing:

SEQ ID NO: 1 is the nucleotide sequence of miR-151-5p.
SEQ ID NO: 2 is the nucleotide sequence of miR-451 mimic
SEQ ID NO: 3 is the nucleotide sequence of miR-1290.
SEQ ID NO: 4 is the nucleotide sequence of a miR-1290 mimic
SEQ ID NO: 5 is the nucleotide sequence of a miR-1290 inhibitor.

### DETAILED DESCRIPTION

### I. Terms

Unless otherwise noted, technical terms are used according to conventional usage, which for example can be found in Benjamin Lewin, Genes V, published by Oxford University Press, 1994 (ISBN 0-19-854287-9); Kendrew et al. (eds.).

Acute lymphoblastic leukemia (ALL) is a cancer of the lymphoid line of blood cells characterized by the development of large numbers of immature lymphocytes. Symptoms may include feeling tired, pale skin color, fever, easy bleeding or bruising, enlarged lymph nodes, or bone pain. As an acute leukemia, ALL progresses rapidly and is typically fatal within weeks or months if left untreated.

In most cases, the cause is unknown. Genetic risk factors may include Down syndrome, Li-Fraumeni syndrome, neurofibromatosis type 1 and/or specific chromosomal translocation. Environment risk factors may include significant radiation exposure or prior chemotherapy. The underlying mechanism involves multiple genetic mutations that result in rapid cell division. The excessive immature lymphocytes thus produced in the bone marrow interfere with the production of new red blood cells, white blood cells, and platelets. Diagnosis is typically based on blood tests and bone marrow examination.

ALL is typically treated initially with chemotherapy aimed at bringing about remission. This is then followed by further chemotherapy typically over a number of years. Additional treatments may include intrathecal chemotherapy or radiation therapy if spread to the brain has occurred. Stem cell transplantation may be used if the disease recurs following standard treatment.

ALL occurs most commonly in children, particularly those between the ages of two and five. In children with ALL, too many stem cells become lymphoblasts, B lymphocytes, or T lymphocytes, which are cancerous and do not function like normal lymphocytes. Blasts are precursors to the mature, circulating blood cells such as neutrophils, monocytes, lymphocytes and erythrocytes. Normally, blasts are usually found in low numbers in the bone marrow and are not usually found in significant numbers in the blood. Circulating blasts can be seen with severe infections, medications (e.g. granulocyte colony stimulating factor), bone marrow replacing processes and hematopoietic neoplasms, of which acute leukemia is the most important hematopoietic neoplasm to recognize because it can rapidly lead to death.

The most common type of ALL is precursor B-lymphoblastic leukemia, also interchangeable termed herein “B-cell ALL”, “B-lineage ALL”, “precursor B-cell ALL”, “B-cell precursor ALL” or “B-cell acute lymphocytic leukemia”. ALL is notable for being the first disseminated cancer to be cured. Survival for children increased from under 10% in the 1960s to 90% in 2015. Survival rates remain lower for infants (50%) and adults (35%). Early relapse rather than excessive toxic complications has been identified as the major factor responsible for the poor outcome in infant ALL.

Certain factors affect prognosis (chance of recovery) and treatment options in pediatric ALL. The prognosis depends, for example, on how quickly and how low the leukemia cell count drops after the first month of treatment; age at the time of diagnosis, sex, race, and ethnic background; the number of white blood cells (WBCs) at the time of diagnosis; whether the leukemia cells are B lymphocytes or T lymphocytes; whether there are certain changes in the chromosomes of the cancerous lymphocytes; whether the child has Down syndrome; whether leukemia cells are found in the cerebrospinal fluid; and the child's weight at the time of diagnosis and during treatment.

The early response to initial prednisone treatment, also referred to herein and in the art as “prednisone response”, is an established predictive factor for treatment outcome in childhood ALL, treated according to the ALL-Berlin-Frankfurt-Münster (ALL-BFM) treatment protocols. Prednisone is a corticosteroid drug commonly used to treat many inflammatory conditions. In current BFM trials for ALL, therapy for all patients starts with a 7-day monotherapy with prednisone and one intrathecal dose of methotrexate on day 1. The dosage of prednisone is increased steadily from the first day of its administration (dl of treatment). On day 8 (d8), the number of leukemic blasts in the blood is calculated. Prednisone response is defined as good, if the peripheral blast count is <1000 blasts/μl, and poor, if >1000 blasts/μl on d8. Prednisone good responders are considered as having a median 8-year event-free survival (EFS) of 82% in contrast to prednisone poor responders, having an EFS of only 34%. For convenience, prednisone response (i.e., blast count) at day 8 is sometimes referred to herein simply as “d8”.

Treatment options depend, for example, on whether the leukemia cells began from B lymphocytes or T lymphocytes; whether the child has standard-risk, high-risk, or very high-risk ALL; the age of the child at the time of diagnosis; whether there are certain changes in the chromosomes of lymphocytes, such as chromosomal translocation, for example, the Philadelphia chromosome in Philadelphia chromosome-positive (Ph+) ALL; whether the child was treated with steroids before starting the induction therapy; and how quickly and how low the leukemia cell count drops during treatment.

For leukemia that relapses (comes back) after treatment, the prognosis and treatment options depend partly, for example, on the time span between first diagnosis and relapse and whether the leukemia relapses in the bone marrow or in other parts of the body.

An important risk factor in ALL is chromosomal translocation, namely, an unusual arrangement of the chromosomes occurring, for example, when two fragments break off from two different chromosomes and swap places (reciprocal translocation), or when one chromosome becomes attached to another (known as Robertsonian translocation). Translocations generate novel chromosomes, places genes in new linkage relationships and/or generate chromosomes without normal pairing partners. Depending on the chromosome breakpoints, a translocation can result in the disruption or misregulation of normal gene function. Approximately 75% of childhood ALL cases harbor recurrent genetic abnormalities, including aneuploidy (the presence of an abnormal number of chromosomes) or structural chromosomal arrangements (e.g., translocations), detected by conventional karyotyping and fluorescence in situ hybridization (FISH). A translocation occurring between the band 21 of the long arm of chromosome 4 and band 23 of the long arm of chromosome 11 [t(4;11)(q21;q23)], which leads to rearrangement of the mixed-lineage leukemia (MLL) gene and generation of the fusion gene MLL-AF4 (this translocation is also herein sometimes designated “MLL-AFF1(AF4)” or “MLL-AF4”), is one of the most recurrent chromosomal aberrations in ALL. Further ALL-related translocations include, for example, translocation [t(9;22)(q34;q11)] generating the fusion gene BCR-ABL1, which is a relatively rare mutation in pediatric ALL, also known as the Philadelphia chromosome-positive (Ph+) mutation; t(12;21)(p13;q22) herein also designated “ETV6-RUNX1” or “TEL-AML1” translocation; and hyperdiploidy (greater than 50 chromosomes). In infants, these translocations are found at the highest frequency in B-ALL, with [MLL-AF4] being related to the poor prognosis. Other recurrent cytogenetic abnormalities include hypodiploidy (42-45 chromosomes) and translocation t(1;19)(q23;p13) herein designated “TCF3-PBX1” or E2A-PBX1”. Advances in cytogenetics have uncovered additional DNA alterations affecting genes involved in normal hematopoiesis, tumor suppression, apoptosis, and cell cycle regulation, including IKZF1, CRLF2, PAX5, and FLT3 genes. Ikaros, the protein coded by the gene IKZF1, is a regulator of lymphoid development, and polymorphisms in the gene have been associated with the childhood ALL. Additionally, IKZF1 deletions and mutations identify high risk biological subsets of ALL.

Cox proportional hazards model or COX-regression model is a statistical regression model that allows to analyze survival with respect to several factors simultaneously, and, optionally, further provides the effect size for each factor. In clinical investigations, there are many situations where several known quantities (covariates), potentially affect patient prognosis. Basic concepts of survival analyses and methods for analyzing and summarizing survival data include, for example, the definition of hazard and survival functions; the construction of Kaplan-Meier survival curves for different patient groups; and the logrank test for comparing two or more survival curves. Kaplan-Meier curves and logrank tests are examples of univariate analysis. They describe the survival according to one factor under investigation, but ignore the impact of any others. Kaplan-Meier curves and logrank tests are useful only when the predictor variable is categorical (e.g.: treatment A vs treatment B; males vs females). They don't work easily for quantitative predictors such as gene expression, weight, or age.

The Cox proportional hazards model is one of the most important methods used for modelling survival analysis data. This analysis works for both quantitative predictor variables and for categorical variables, and assesses simultaneously the effect of several risk factors on survival time. The Cox model is expressed by the hazard function that can be interpreted as the risk of dying at time t, and is estimated as follow:

h(t)=h₀(t)×exp(b₁x₁+b₂x₂+ . . . +bₚxₚ)

where, h(t) is the hazard function determined by a set of p covariates (x₁, x₂, . . . , xₚ) that may vary over time; the coefficients (b₁, b₂, . . . , bₚ) measure the impact (i.e., the effect size) of the covariates; the term h₀ is the baseline hazard, and corresponds to the value of the hazard if all the xᵢ are equal to zero (the quantity exp(0) equals 1).

The quantities exp(bᵢ) are termed herein “hazard ratios” (HR). A value of bᵢ greater than zero, or equivalently a hazard ratio greater than one (HR>1), indicates that as the value of the i^{th} covariate increases, the event hazard increases and thus the length of survival decreases. Accordingly, when HR<1 the event hazard reduces (i.e., reduction in the hazard), and when HR=1 the covariate has no effect on survival.

Minimal residual disease. Minimal residual disease (MRD) monitoring has high prognostic value in childhood ALL. This method has been developed and standardized in Europe and became essential to large-sized multicenter clinical trials. As a strong correlation between the MRD levels at an early stage of therapy and clinical outcome on various cases has been recognized, many trials have incorporated the stratification according to the amount of MRD. Based on the sensitive measurement of early response to cytotoxic treatment, it is possible to identify not only patients at high risk for relapse but also a group of low-risk patients with an excellent relapse-free survival (RFS) of more than 95%. Hence, MRD information provides a definition of remission in childhood ALL, and MRD data are incorporated in current treatment protocols to refine risk assignment. MRD quantification is based on real time (quantitative) polymerase chain reaction (qPCR) amplification of certain immunoglobulin (Ig) and/or T-cell receptor (TCR) gene rearrangements as targets. MDR detection by qPCR is also referred to herein as “polymerase chain reaction-based minimal residual disease quantification”, “qPCR-based MRD assay”, or simply as “PCR-MRD”. These gene rearrangements can easily be identified in most patients at diagnosis with limited sets of PCR primers. Moreover, using these molecular targets, sensitivities of 10⁻⁴ to 10⁻⁶ (1 malignant cell within 10⁴ to 10⁶ normal cells) are obtained routinely.

Nicotinamide phosphoribosyltransferase (NAMPT) is a regulator of the intracellular nicotinamide adenine dinucleotide (NADH or its oxidized form NDA⁺ sometimes collectively referred to herein as “NAD”) pool. The enzyme NAMPT catalyzes the condensation of nicotinamide with 5-phosphoribosyl 1-pyrophosphate to yield nicotinamide mononucleotide, one step in the biosynthesis of NADH. NADH is an essential coenzyme involved in cellular redox reactions and is a substrate for NADH/NAD⁺-dependent enzymes. Through its NADH-biosynthetic activity, NAMPT influences the activity of NADH/NAD⁺-dependent enzymes, thereby regulating cellular metabolism. NAMPT has a crucial role in cancer cell metabolism, is often overexpressed in tumour tissues and is an experimental target for anti-tumour therapies. One inhibitor of NAPMT is the small molecule FK866 which competes for the same binding site as nicotinamide, but due to its very low dissociation rate, it is essentially an irreversible inhibitor.

New born, infant, child, adolescent. As referred to herein, “newborn” usually refers to a human baby from birth to about 2 months of age; “infant” is a human individual anywhere from birth to 1 year old, “child” is young individual who is not yet an adult, and in the context of some embodiments, a child is 1 to 15 years old; adolescent is a human aged between 15 and 19, inclusive. The term “pediatric” as referred to herein, related to a branch of medicine concerned with diseases of infants, children, and adolescents.

Abnormal. Deviation from normal characteristics is referred to herein as abnormal. Normal characteristics can be found in a control, a standard for a population, and the like. For instance, where the abnormal condition is a disease condition, such as ALL, a few appropriate sources of normal characteristics might include an individual who is not suffering from the disease, or a population who did not experience a particular prognosis outcome of the disease, such as ALL relapse. Similarly, abnormal may refer to a condition that is associated with a disease or disease relapse. The term “associated with” includes an increased risk of developing the disease or a relapse thereof. For instance, a certain abnormality (such as an abnormality in expression of a miRNA) can be described as being associated or correlated with the biological condition of ALL relapse. Controls or standards appropriate for comparison to a sample, for the determination of abnormality, such as in the determination of an expression cutoff value, include samples believed to be normal as well as laboratory-determined values, even though such values are possibly arbitrarily set, and keeping in mind that such values may vary from laboratory to laboratory. Laboratory standards and values may be set based on a known or determined population value and may be supplied in the format of a graph or table that permits easy comparison of measured, experimentally determined values.

Active agent, pharmaceutical agent. The terms “active agent”, “active ingredient”, “pharmaceutical agent” and “active pharmaceutical ingredient (API)” as used herein are interchangeable and refer to a compound, e.g., a chemical compound, or a composition of at least two compounds, e.g., a complex or conjugate, which is accountable for a desired biological or chemical effect such as a desired therapeutic or prophylactic effect, when properly administered to a subject or a cell. An active compound may exert its biological effect via, for example, direct or indirect contact with a target, wherein “contacting” includes incubating with, or otherwise exposing the active agent in solid or in liquid form to a target, e.g., a cell, for a sufficient period of time for the agent to interact with the cell.

In the context of embodiments described in the present disclosure, the active agent may be, for example, miR-451 or FK866 when used as inhibitors of NAMPT.

Administration. The introduction of an active compound and/or a composition into a subject by any route known to one of skill in the art, is referred to herein as administration. Administration can be local or systemic. Examples of local administration include, but are not limited to, topical administration, subcutaneous administration, intramuscular administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Analog, derivative, mimetic. An “analog”, as referred to herein, is a molecule that differs in chemical structure from a parent compound, for example a homolog (differing by an increment in the chemical structure, such as a difference in the length of an alkyl chain), a molecular fragment, a structure that differs by one or more functional groups, and/or a change in ionization. Structural analogs are often found using quantitative structure activity relationships (QSAR), with techniques known in the art. A derivative is a biologically active molecule derived from the base structure. A mimetic is a molecule that mimics the activity of another molecule, such as a biologically active molecule, for example a peptide. Biologically active molecules can include chemical structures that mimic the biological activities of a compound. Such biologically active molecules are exemplified by peptidomimetics. It is acknowledged that these terms may overlap in some circumstances.

Antagonist. As referred to herein, “antagonist” is a molecule or compound that tends to nullify the action of another, or in some instances that blocks the ability of a given substrate e.g., a chemical, to bind to its receptor, or other interacting molecule, thus preventing a biological response. Antagonists are not limited to a specific type of compound, and may include, in various embodiments, peptides, antibodies and fragments thereof, and other organic or inorganic compounds (for example, peptidomimetics and small molecules).

Antibody. A polypeptide ligand comprising at least a light chain or heavy chain immunoglobulin variable region, which specifically recognizes and binds an epitope of an antigen such as the NAMPT or JAK2 protein or a fragment thereof. Antibodies are composed of a heavy chain and a light chain, each of which has a variable region, termed the variable heavy (VH) region and the variable light (VL) region, respectively. Together, the VH region and the VL region are responsible for binding the antigen recognized by the antibody. The term “antibody”, as used herein, includes an intact immunoglobulin as well as a variant and portion thereof well known in the art, such as a Fab′ fragment, F(ab)′2 fragment, single chain Fv protein (“scFv”), and disulfide stabilized Fv protein (“dsFv”). The term also includes recombinant forms such as chimeric antibodies (for example, humanized murine antibodies), heteroconjugate antibodies (such as bispecific antibodies).

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Antisense inhibitor. Refers to an oligomeric compound that is at least partially complementary to the region of a target nucleic acid molecule to which it hybridizes, e.g., in order to block it. As used herein, an antisense inhibitor (also referred to as an “antisense compound”) that is “specific for” a target nucleic acid molecule, is one which specifically hybridizes with, and modulates expression of, the target nucleic acid molecule. Non-limiting examples of antisense compounds include primers, probes, antisense oligonucleotides, small interfering RNAs (siRNAs), micro RNAs (miRNAs), short hairpin RNAs (shRNAs) and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self complementarity to allow for hybridization and formation of a fully or partially double-stranded compound.

Biological Sample. Any sample that may be obtained directly or indirectly from an organism is referred to herein as a biological sample, including, for example, bone marrow, whole blood, plasma, serum, tears, mucus, saliva, urine, pleural fluid, spinal fluid, gastric fluid, sweat, semen, vaginal secretion, sputum, fluid from ulcers and/or other surface eruptions, blisters, abscesses, tissues, cells (such as, fibroblasts, peripheral blood mononuclear cells, or muscle cells), organs, and/or extracts of tissues, cells or organs. A sample is collected or obtained using methods well known to those skilled in the art.

Complementary DNA (cDNA). A piece of DNA lacking internal, non-coding segments (introns) and transcriptional regulatory sequences. cDNA can contain untranslated regions (UTRs), such as those that are responsible for translational control in the corresponding RNA molecule. cDNA is synthesized in the laboratory, for example, by reverse transcription from RNA extracted from cells.

Contacting. Placement in direct physical association, both in solid and liquid form. Contacting can occur in vitro with isolated cells or in vivo by administering to a subject.

Control. A reference standard is referred to herein as “control”. A control can be a known value indicative of basal expression of a diagnostic molecule such as miR-1290 or miR-451 described herein. In particular examples, a control sample is taken from a subject that is known not to have a disease or condition, including ALL patients who did or did not experience disease relapse. In other examples a control is taken from the subject being diagnosed, but at an earlier time point, either before disease onset or prior to or at an earlier time point in disease treatment. A difference between a test sample and a control can be an increase or, conversely, a decrease. The difference can be a qualitative difference or a quantitative difference, for example a statistically significant difference. In some embodiments, a difference is an increase or decrease, relative to a control, of at least about 10%, for example, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Correlate, correlating. The terms “correlate” and “correlating”, as used herein, refer to an action by which a relationship is set forth between two phenomena so as to establish a mutual or reciprocal relation between a first phenomenon that accompanies a second phenomenon, which is usually parallel to it, and is related in some way to it. To correlate is to bear reciprocal or mutual relations. For example, expression levels of miR-451 are correlated with expression of NAMPT such that a change in miR-451 expression level results in an opposite change in NAMPT expression.

Detect, detection. Detection is determining if an agent (such as a signal or particular nucleic acid probe) is present or absent. In some embodiments, this can further include quantification.

Determining expression of a gene product. Detection of a level of expression (for example of a nucleic acid) in either a qualitative or a quantitative manner. In one example, determining expression of a gene product is the detection of a miRNA, and/or of the corresponding protein as described herein.

Diagnosis. The process of identifying a disease or a predisposition to developing a disease or condition by its signs, symptoms, and results of various tests and methods, is referred to herein as “diagnosing”. The conclusion reached through that process is called “diagnosis”. For example, ALL and/or its relapse may be diagnosed, for example, by the methods disclosed herein. The term “predisposition” refers to an effect of a factor or factors that render a subject susceptible to, or at risk for, a condition, disease or disorder, such as ALL and/or its relapse. In contemplated methods described herein, specific miRNA expression determination is used to identify a subject predisposed to (or at an increased risk for) ALL relapse.

Effective amount. An “effective amount” of, for example, a compound (e.g., an active agent), or a composition comprising it, as referred to herein, is a quantity of the compound or composition sufficient to achieve a desired effect in a subject being treated. An effective amount of a compound or a composition can be administered in a single dose or in several doses, for example, daily, during a course of treatment. The effective amount of the compound and/or composition will be dependent on the compound applied, the subject being treated, the severity and type of the affliction, and the manner of administration of the compound or composition.

Expression clone. Expression clone is a plasmid in bacteria or a phage in bacteria, or a vector in a host cell, designed to produce a protein from a fragment of DNA insert.

Expression Control Sequences. Nucleic acid sequences that regulate the expression of a homologous or heterologous nucleic acid sequence are termed herein “expression control sequences”. Expression control sequences are operatively linked to, or are an integral part of, a nucleic acid sequence such that they control and regulate the transcription and, as appropriate, translation of the nucleic acid sequence. A non-limiting example of an expression control sequence operatively linked to a nucleic acid sequence being controlled is a miRNA. For example, the expression of NAMPT is being controlled by miR-451. Expression control sequences that are integral parts of a nucleic acid sequence include, for example, appropriate promoters, enhancers, transcription terminators, a start codon (ATG) in front of a protein-encoding gene, splicing signal for introns, sequences that afford maintenance of the correct reading frame of a gene to permit proper translation of mRNA, and stop codons. For example, a polynucleotide coding a gene can be inserted into an expression vector that contains a promoter sequence, which facilitates the efficient transcription of the inserted genetic sequence by the host.

An expression control sequence is further exemplified by the three prime untranslated region (3′-UTR), which is the section of mRNA that immediately follows the translation termination codon. The 3′-UTR often contains regulatory regions that post-transcriptionally influence gene expression. Regulatory regions within the 3′-untranslated region contain both binding sites for regulatory proteins as well as for miRNAs, which can influence polyadenylation, translation efficiency, localization, and stability of the mRNA. microRNA response elements (MREs) are sequences in the 3′-UTR to which miRNAs specifically bind and, thereby, can decrease gene expression by various mRNAs by either inhibiting translation or directly causing degradation of the transcript. Exemplary embodiments described herein pertain to regulation of NAMPT gene expression effected by binding of miRNA-451 to 3′-UTR of NAPMT mRNA.

Increased risk. As used herein “increased risk” of ALL relapse refers to an increase in the statistical probability of an ALL patient relapsing relative to the general population, following standard disease treatment. As described herein, the risk of a subject determined to have an increased risk of ALL relapse may be a high risk or intermediate risk, both of which are an increased risk in comparison to “standard risk”.

Inhibiting protein activity. Herein, inhibition of protein activity is decreasing, limiting, or blocking an action, function and/or expression of a protein. The phrase “inhibit protein activity” is not intended to be an absolute term. Instead, the phrase is intended to convey a wide-range of inhibitory effects that various agents may have on the normal (for example, uninhibited or control) protein activity. Inhibition of protein activity may, but need not, result in an increase in the level or activity of an indicator of the protein's activity. By way of example, this can happen when the protein of interest is acting as an inhibitor or suppressor of a downstream indicator. Thus, protein activity may be inhibited when the level and/or activity of any direct or indirect indicator of the protein's activity is changed (for example, increased or decreased) by at least 10%, at least 20%, at least 30%, at least 50%, at least 80%, at least 100% or at least 250% or more, as compared to control measurements of the same indicator.

Isolation. An isolated biological component (such as a nucleic acid molecule, protein or organelle), as referred to herein, is a biological component that has been substantially separated or purified away from other biological components in the cell of the organism in which the component naturally occurs, for example, other chromosomal and extra-chromosomal DNA and RNA, proteins and organelles. Nucleic acids and proteins that have been isolated include nucleic acids and proteins purified by standard purification methods.

The term “isolated” as used herein also embraces nucleic acids and proteins prepared by recombinant expression in a host cell as well as chemically synthesized nucleic acids.

Label. A detectable compound or composition that is conjugated directly or indirectly to another molecule to facilitate detection of that molecule, is referred to herein as “label”. Specific, non-limiting examples of labels include radioactive isotopes, enzyme substrates, co-factors, ligands, chemiluminescent or fluorescent agents, haptens, and enzymes.

microRNA (miRNA). microRNA, designated herein “miRNA” or “miR”, is a short, non-coding single-stranded RNA molecule of 18-24 nucleotides long. Various miRNAs are widely conserved in all eukaryotic organisms and serve as regulators of gene expression (e.g., as expression control sequences). miRNAs can inhibit translation, or can direct cleavage of target mRNAs through complementary or near-complementary hybridization to a target nucleic acid. miRNAs are involved in all major cellular processes and are implicated in a large number of human diseases including cancer.

miRNAs are endogenously transcribed in cells from longer precursor molecules of DNA by RNA polymerase II. This enzyme produces capped and polyadenylated primary transcripts (termed “pri-miRNAs”) that can be either protein-coding or non-coding. A primary transcript is cleaved by the Drosha ribonuclease III enzyme to produce an approximately 70-nucleotide stem-loop precursor miRNA (termed “pre-miRNA”), which is further cleaved by the cytoplasmic Dicer ribonuclease to generate the mature miRNA. Mature miRNA is incorporated into a RNA-induced silencing complex (RISC), which recognizes target mRNAs through imperfect base pairing with the miRNA and this most commonly results in translational inhibition or destabilization of the target mRNA.

The numbering method of miRNA genes is simply sequential, corresponding to the order of their first publication. The name/identifier in the database, for example, of the miR-451 gene is of the form “hsa-mir-451”, wherein the first three letters signify the organism, and in this exemplary gene “hsa” stands for homo sapiens, signifying its human source. The number “451” is the serial publication number of the gene. mir-451 refers also to the predicted stem-loop portion of the primary transcript. The mature miRNA is designated in the database, for example, as “hsa-miR-451” or simply “miR-451”. Distinct precursor sequences and genomic loci that express identical mature miR sequences get names of the form, for example, hsa-mir-121-1 and hsa-mir-121-2. Lettered suffixes denote closely related mature sequences, for example, hsa-miR-121a and hsa-miR-121b would be expressed from precursors hsa-mir-121a and hsa-mir-121b, respectively.

Sometimes two ˜22-nucleotide sequences miRNAs originate from the same precursor, i.e, each corresponds to one of the two complementary strands of the precursor DNA. When the relative abundancies clearly indicate which is the predominantly expressed miRNA, the mature sequences are assigned names of the form, for example, of “miR-56” (the predominant product) and “miR-56*” (from the opposite arm of the precursor). When the data are not sufficient to determine which sequence is the predominant one, names like “miR-151-5p” (from the 5′ arm) and “miR-151-3p” (from the 3′ arm) prevail. Herein, for convenience, “miR-151-5p” and “miR-151” are interchangeable but they both refer to miR-151-5p.

As used herein, a “microRNA sequence” includes both mature miRNA sequences as well as precursor sequences, e.g., pri-miRNA and pre-miRNA.

microRNA mimic, agomir, inhibitors and antagomirs (antagomiRNAs). Synthetic miRNA mimics and agomirs are double-stranded miRNA-like RNAs (dsmiRNAs), which are designed to copy the functionality of mature endogenous miRNA. Upon transfection thereof to cells, a miRNA mimic can regulate the biological function of a target gene by mimicking endogenous microRNA.

A synthetic microRNA inhibitor (also referred to herein as “antisense”, “antisense miR” or “antisense miRNA”) and antagomirs (also denoted herein “antagomiRNAs” or “antagomiRs”) are single-stranded oligonucleotides, fully complementary, namely, antisense, to their target endogenous mature miRNA. Antagomirs/miRNA inhibitors silence their corresponding mature miRNA and inhibit its expression by binding thereto, thus effectively preventing the target miRNA from binding to normal cellular binding sites. Non-limiting examples of antisense miR inhibitors include vector-based expression clones of miRNA inhibitors commercially known as miArrest™. These miRNA inhibitor constructs bind specifically to their target miRNA upon transduction into cells. The post-transcriptional processing causes formation of an entrapping structure (kind of a “hole” in a hairpin structure) that attracts and binds two molecules of the intended endogenous miRNA, thereby preventing the binding of miRNA to its target mRNA. A further non-limiting example of antisense miRNA inhibitor is miR-1290 antisense designated herein as SEQ ID NO:5.

Antagomirs differ from inhibitors (antisense miR) in that they are chemically-modified to contain one or more of (i) 2′-methoxy throughout the entire antisense strand; (ii) 2 phosphorothioates at the 5′ end; and (iii) 4 phosphorothioates plus 4 cholesterol moieties at the 3′ end. AntagomiRs, the synthetic 2-O-methyl RNA oligonucleotides, have a stronger binding to the miRNA-associated gene silencing complexes (RISCs) than endogenous mature miR, thus, they effectively compete with miRNA on binding to a target mRNAs. Non-limiting examples of antagomiRNAs include antagomiR-451, and antagomiR-1290.

Agomirs differ from miR mimics in that agomirs contain similar chemical modifications as antagomirs, thus, being synthetic 2-O-methyl RNA oligonucleotides. Agomirs and antagomirs exhibit enhanced transfection efficiency and increased resistance to various RNases. Overexpression studies may be performed, e.g., by using vectors comprising agomirs or dsmiRNAs (mimics) that “mimic” mature miRNA. miRNA mimics are exemplified herein by synthetic miR-451 mimic and synthetic miR-1290 mimic designated herein as SEQ ID NOs:2 and 4, respectively.

miRNA mimics, antagomiRs, miRNA inhibitors and agomirs are commercially available as vector-based expression clones or synthetic oligonucleotides, or can be chemically synthesized. For example, antagomiRs may be synthesized with 2′-OMe modified bases (i.e., 2′-hydroxyl of the ribose is replaced with a methoxy group), phosphorothioate (phosphodiester linkages are changed to phosphorothioates) on the first two and last four bases, and an addition of cholesterol motif at 3′ end through a hydroxyprolinol modified linkage.

Scrambled miRNA. Scrambled miRNAs (or scrambled miRs), also referred to herein as “miRNA mimic negative control” are validated random sequences which have been tested on mammalian cells and tissues and are shown to produce no identifiable effects on known miRNA function. These oligonucleotides comprise a scrambled, non-targeting stem-loop sequence of a precursor miRNA either of the same corresponding mature miRNA tested or a universal sequence, allowing to easily control for increased miRNA effects of interest. In some embodiments described herein, universal oligonucleotides are used as negative control in miRNA mimic experiments, optionally provided to the cells as non-targeting pre-miRNA lentivectors that further express the targeted miRNAs, e.g., miR mimics.

Normalization. As referred to herein, “normalization” is a process by which data are corrected for factors other than those being directly tested in the experiment. For example, to normalize reporter data, the reporter activity in a particular sample is divided by a second value specific to the same sample. The primary purpose of normalization is to remove sample-to-sample variability caused by factors other than those being tested in the experiment. These factors can include, for example, variabilities in cell plating and transfection efficiency, pipetting inconsistencies, and toxicity. Data from each sample is normalized prior to making comparisons between test groups, thereby reducing variability and allowing data comparisons to be made with greater confidence. Non-limiting methods for normalization include normalization to total protein content, total ATP content or cell number, and normalization to a control reporter vector. In cell transfection studies, protein normalization can tighten reporter assay results and may be useful when using stably transfected cells.

In reporter assays using transiently transfected cells, significant variability can be introduced during transfection, and in such cases vector normalization is preferably performed. Vector normalization is accomplished by co-transfection of a control vector, often referred to as an “internal vector control”, along with the test vector. The internal vector control has a constitutively active promoter driving expression of a second (control) reporter protein. Control reporter protein activity correlates to the amount of DNA transfected into the cells and the general ability of the cells to express protein. Reporter activity from this internal control is assayed along with the test reporter and used to normalize the test reporter data. By factoring in transfection efficiency, vector normalization reduces data variability and can give differences between test groups greater statistical significance. A promoter for the control vector will ideally give low to medium reporter expression and consistent expression under the experimental conditions being tested. Non-limiting examples of promoters include TK, SV40 and cytomegalovirus (CMVO promoters.

Is some embodiments, luciferases are used as genetic reporters in transiently co-transfected cells. In accordance with these embodiments, for vector normalization, activity of two luciferases, for example, Firefly and Renilla, are measured in the same cells or lysate aliquot. In exemplary embodiments, Firefly luciferase is used as the test reporter and Renilla luciferase as the control reporter.

Oligonucleotide. As used herein, “oligonucleotide” refers to a plurality of joined nucleotides, between about 6 and about 300 nucleotides in length. An oligonucleotide analog refers to a subclass of oligonucleotides that contain moieties that function similarly to oligonucleotides but have non-naturally occurring portions. For example, oligonucleotide analogs can contain non-naturally occurring portions such as altered sugar moieties or inter-sugar linkages, such as a phosphorothioate oligodeoxynucleotide, or can comprise peptide nucleic acid (PNA) molecules. Functional analogs of naturally occurring polynucleotides can bind to RNA or DNA. Particular oligonucleotides and oligonucleotide analogs can include linear sequences up to about 200 nucleotides in length, for example, a sequence (such as DNA or RNA) that is at least 6 bases, for example at least 8, 10, 15, 20, 25, 30, 35, 40, 45, 50, 100 or even 200 bases long, or from about 6 to about 50 bases, for example from about 10 to about 25 bases, 12, 15 or 20 bases.

Pharmaceutical composition. The term “pharmaceutical composition”, as used herein, refers to a formulation designed for medicinal utilization such as, but not limited to, therapeutic or diagnostic utilization. “Formulation” as used herein refers to any mixture of different components or ingredients prepared in a certain way, i.e., according to a particular formula. For example, a formulation may include one or more drug substances, active agents or active pharmaceutical ingredients (APIs) combined or formulated together with, for example, one or more carriers, excipients, stabilizers and the like. The formulation may comprise solid and/or non-solid, e.g., liquid, gel, semi-solid (e.g. gel, wax) or gas components. Usually, in a formulation for pharmaceutical administration the APIs are combined or formulated together with one or more pharmaceutically and physiologically acceptable carriers, which can be administered to a subject (e.g., human or non-human subject) in a specific form, such as, but not limited to, tablets, linctus, ointment, infusion or injection.

Pharmaceutically acceptable carriers are approved (e.g., by a regulatory agency of the Federal or a state government or listed in the U.S. Pharmacopeia or other generally recognized pharmacopeia for use in animals, and more particularly in humans) carriers, vehicles, or diluents that do not cause significant irritation to an organism and do not abrogate the biological activity and properties of an active agent. Physiologically suitable carriers in liquid formulations may be, for example, solvents or dispersion media. The use of such media and agents in combination with pharmaceutically active agents is well known in the art. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol and the like as a vehicle. For solid compositions (for example, powder, pill, tablet, or capsule forms), conventional non-toxic solid carriers can include, for example, pharmaceutical grades of mannitol, lactose, starch, or magnesium stearate.

Pharmaceutically acceptable excipients. Herein the term “excipient” refers to an inert, non-toxic auxiliary substance added to a pharmaceutical composition (formulation) to further facilitate process and administration of the active ingredients. Pharmaceutically acceptable excipients encompass wetting or emulsifying agents, preservatives, antioxidants, coatings, isotonic, absorption delaying agents, pH buffering agents and the like, which are approved for use in animals, and more particularly in humans.

Preventing or treating a disease. Preventing a disease refers to inhibiting the full development of a disease, for example inhibiting the progression or metastasis of a tumor in a subject with a neoplasm. Treating a disease, as referred to herein, means ameliorating, inhibiting the progression of, delaying worsening of, and even completely preventing the development of a disease. Treatment refers to a therapeutic intervention that ameliorates a sign or symptom of a disease or a pathological condition after it has begun to develop. In particular examples, however, treatment is similar to prevention, except that instead of complete inhibition, the development, progression or relapse of the disease is inhibited or slowed. In particular embodiments, a treatment will decrease the probability that a condition, for example, ALL relapse, will develop.

PCR Amplification. When used in reference to a nucleic acid, any technique that increases the number of copies of a nucleic acid molecule in a sample or specimen is amplification. An example of amplification technique is the polymerase chain reaction (PCR, in all of its forms), in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed to new templets, extended, and dissociated so as to amplify the number of copies of the nucleic acid. The product of in vitro amplification can be characterized by electrophoresis, restriction endonuclease cleavage patterns, oligonucleotide hybridization or ligation, and/or nucleic acid sequencing, using standard techniques.

Real time PCR also termed herein “quantitative PCR” or “qPCR” is a method for detecting, characterizing and quantifying DNA products generated during each cycle of a PCR amplification, which products are proportionate to the amount of template nucleic acid present prior to the start of PCR. The information obtained, such as an amplification curve, are used to quantitate the initial amounts of template nucleic acid sequence.

Real-time PCR combines PCR amplification and detection into a single step. This eliminates the need to detect products using gel electrophoresis and, more importantly, it enables the method to be truly quantitative. As in standard PCR, DNA is amplified by 3 repeating steps, each step being effected at a distinct temperature range (each PCR cycle is hence also referred to as a “thermal cycle”): denaturation, annealing and elongation. However, in qPCR, fluorescent dyes are used to label PCR products during thermal cycling, whereby during each cycle, the fluorescence is measured, enabling the collection of data as PCR progresses. Real-time PCR instruments measure the accumulation of fluorescent signal during the exponential phase of the reaction for fast, precise quantification of PCR products and objective data analysis.

In a real-time PCR assay, a positive reaction is detected by accumulation of a fluorescent signal. The cycle threshold (Ct) is defined as the number of cycles required for the fluorescent signal to cross the threshold (i.e. exceed background level). Ct levels are inversely proportional to the amount of target nucleic acid in the sample, i.e., the lower the Ct level, the greater the amount of target nucleic acid in the sample, wherein Cts <29 are strong positive reactions indicative of abundant target nucleic acid in the sample. A “delta-Ct”, as referred to herein, is the difference between Ct specific to the sequence of interest and Ct of a reference sequence, usually the sequence of an abundant “house-keeping gene” which thus uses as a normalization means (particularly where the target sequence does not have the same concentration in all samples tested. Differences between Ct values of two or more samples may be due, e.g., to a different amount of biological material or different number of cells). The reference sequence, also referred to herein as “reference gene” may be one or more constantly expressed genes, for example, but not limited to, 5S Ribosomal RNA.

Reverse transcription PCR (RT-PCR) allows the detection and amplification of RNA templates. The RNA is reverse transcribed into complementary DNA (cDNA), using reverse transcriptase. The first step of RT-PCR is the synthesis of a DNA/RNA hybrid. The single stranded DNA molecule is then completed by the DNA-dependent DNA polymerase activity of the reverse transcriptase into cDNA. Reverse transcriptase also has an RNase function, which degrades the RNA portion of the hybrid. From here on, the standard PCR procedure is employed to amplify the cDNA. The possibility to revert RNA into cDNA by RT-PCR has many advantages. Most commonly, it serves as a first step in qPCR, which quantifies RNA transcripts in a biological sample, and allows the detection of low abundance RNAs in a sample.

Quantitative reverse transcription PCR (RT-qPCR) also referred to herein as “real time RT-PCR”, allows the detection, amplification and quantification of RNA templates. RNA is first transcribed into cDNA by reverse transcriptase from total RNA or messenger RNA (mRNA). The cDNA is then used as the template for the qPCR reaction as described above.

Probes and primers. A probe, as referred to herein, comprises an isolated nucleic acid attached to a detectable label as described herein, or a reporter molecule. Primers are short nucleic acid molecules, preferably DNA oligonucleotides of 10 nucleotides or more in length. Longer DNA oligonucleotides can be about 15, 17, 20, or 23 nucleotides or more in length. Primers can be annealed to a complementary target DNA strand by nucleic acid hybridization to form a hybrid between the primer and the target DNA strand, and then the primer is extended along the target DNA strand by a DNA polymerase enzyme. Primer pairs can be used for amplification of a nucleic acid sequence, e.g., by the PCR or other nucleic-acid amplification methods known in the art.

One of ordinary skill in the art will appreciate that the specificity of a particular probe or primer increases with its length. Thus, in order to obtain greater specificity, probes and primers can be selected that comprise at least 17, 20, 23, 25, 30, 35, 40, 45, 50 or more consecutive nucleotides of the target sequence being amplified. Probes and/or PCR primer pairs are available commercially and/or can be derived from a known sequence, for example, by using computer programs intended for that purpose.

In some embodiments, the primers used are oligonucleotide analogues comprising modified DNA nucleotides in which the 2′-O and 4′-C atoms of the ribose are joined through a methylene bridge. This additional bridge limits the flexibility normally associated with the ring, essentially locking the structure into a rigid bicyclic formation. These oligonucleotide analogs are also referred to herein as “locked nucleic acids” or “LNAs”. When incorporated into qPCR probes or primers, LNA increases thermal duplex stability and improves the specificity of probe hybridization to its target sequence as compared to native-state DNA bases, wherein increasing the number of LNA bases in a qPCR probe increases the hybrid stability and its melting temperature (Tₘ). Primers and probed containing LNA are commercially available (e.g., (LNA® primers of Qiagen).

Prognosis. As used herein, the term “prognosis” refers to a prediction of the course or outcome of a disease or disorder, namely, predicting the likely or expected development of a disease, including whether the signs and symptoms will improve or worsen (and how quickly) or remain stable over time. Prognosis is also construed as the chances of recovery from a disease. As used herein, prognosis also means predicting the efficiency or outcome of a treatment modality or treatment protocol. A prognosis is made on the basis of the normal course of the diagnosed disease, the individual's physical and mental condition, the available treatments, and additional factors.

Recombinant DNA (rDNA). Deoxyribonucleic acid (DNA) molecules formed by laboratory methods of genetic recombination (such as molecular cloning), and comprising genetic material from multiple sources that has been brought together, is referred to herein as “recombinant DNA”. Such an artificially made DNA strand is formed by recombination of two or more gene sequences, wherein the new combination may or may not occur naturally, but is engineered specifically for a particular purpose. Recombinant DNA molecules are also referred to herein as “chimeric DNA” because they can be made of material from two different species.

Small interfering RNAs. “Small interfering RNAs” or “siRNAs”, as referred to herein, are synthetic or naturally-produced small double stranded RNAs (dsRNAs) that can induce gene-specific inhibition of expression in invertebrate and vertebrate species. These interfering or inhibiting dsRNAs are of about 15 to about 40 nucleotides and contain a 3′ and/or 5′ overhang on each strand having a length of 0 to about 5 nucleotides, wherein the sequence of the double stranded RNAs is essentially identical to at least a portion of a coding region of the target gene for which interference or inhibition of expression is desired. The double stranded RNAs can be formed from complementary single stranded RNAs (ssRNAs) or from a ssRNA that forms a hairpin, or from a DNA vector.

Small molecule inhibitor. As used herein, a small molecule inhibitor is a molecule, typically with a molecular weight less than 1000 Daltons or, in some embodiments, less than about 500 Daltons, wherein the molecule is capable of inhibiting, to some measurable extent, an activity of a target molecule.

Treatment modality. The method used to treat a patient for a particular condition. Herein this term is interchangeable with the terms “treatment protocol”, “treatment approach” and “treatment type”.

Vector, plasmid. Plasmid is a small naturally occurring circular DNA element, considered as an extra-chromosomal DNA molecule found mainly in bacteria. This small DNA element carries several genes, but lesser amount than in chromosomal DNA. Plasmids and chromosomes are replicated using the same enzymes, but plasmids are replicated and inherited independently from the bacterial chromosomes. Normally a bacterium will have only one copy of its chromosome, but it can have multiple copies of a plasmid. Plasmids are not essential for the function of bacteria, but these genes give extra survival to bacteria.

A vector is a double-stranded DNA vehicle that carries foreign DNA molecules into host cell. Vectors are mainly used in the recombinant DNA technology to introduce foreign DNA molecules into cells. Vectors can be derived from plasmids (i.e., engineered plasmids). Cosmids, viral vectors, and artificial chromosomes are other types of vectors. Generally, vectors, like plasmids, are extra chromosomal, self-replicative DNA fragments inside a host cell. Vectors are designed for a variety of applications including easy cloning of foreign DNA and easy expression of foreign proteins. A vector can include one or more selectable marker genes and other genetic elements known in the art.

Viral vectors are recombinant DNA vectors (i.e., vectors having recombinant DNA as defined herein) having at least some nucleic acid sequences derived from one or more viruses.

Lentiviral vectors, lentiviral particles. Lentiviruses are retroviruses with long incubation period (months, even years) and a propensity to induce a wide range of pathologies in different animal species. Some examples of lentiviruses are Human immunodeficiency virus (HW), Simian (SW) and Feline (FW) Immunodeficiency Viruses. Due to their rather flexible genome and a potential of transducing many forms of dividing as well as nondividing cells, lentiviruses are widely used viral vectors for gene transfer or delivery. Lentiviruses can deliver large amounts of viral genetic information into the DNA of host cells whereby the viral genome is passed onto daughter cells during division. Lentiviral vectors, also termed herein “lentivectors”, derived from the human HIV-1 are predominantly used for gene delivery in mammalian cells.

Lentiviral particles, also termed herein “pseudoviral particles”, are enveloped lentiviral vectors comprising recombinant DNA. Lentiviral particles are typically produced in HEK 293T cells. Essential lentiviral (e.g., of HW-1) genes must be expressed in these cells to allow the generation of lentiviral particles. These genes are usually expressed by several separated lentivectors and/or engineered plasmids. For example, second generation lentiviral particles are produced by co-transfecting HEK 293T cells with: (i) a lentiviral expression or transfer vector such as pLV-Green, containing the psi (Ψ) packaging sequence and the transgene gene inserted between the lentiviral long terminal repeats (LTRs) allow target cell integration. A human cDNA open reading frames (ORFs) may also be cloned into a lentiviral expression vector; (ii) a packaging vector, such as pLV-HELP, encoding the pol, gag, rev and tat viral genes and containing the rev-response element (RRE); and (iii) an envelope vector or a pseudotyping plasmid, such as pLV-iVSV-G, encoding the G protein of the Vesicular Stomatitis Virus (VSV-G) envelope geneas. Unlike the HW envelope, the VSV-G envelope has a broad cell host range extending the cell types that can be transduced by VSV-G-expressing lentiviruses.

Two days after transfection of HEK 293T cells, the cell supernatant contains lentiviral particles which can be used to transduce desired target cells. Once in the target cells, the viral RNA is reverse-transcribed, imported into the nucleus and stably integrated into the host genome. One or two days after integration of the viral RNA, the expression of the recombinant protein can be detected.

Multiple promoter vectors are lentivectors characterized by the presence of two or more, e.g., three or four genes, at least one of which is the gene of interest and at least one is a marker (e.g., labeling) gene and/or a selection gene, each of these genes being promoted (driven) by an independent mammalian promoter. Non-limiting examples of marker genes include a gene encoding for a fluorescence protein, for example, enhanced green fluorescent protein (EGFP, a variant of wild type green fluorescent protein from a jellyfish), a commonly used green fluorescent protein which ranks high in brightness, monomeric red fluorescent protein 1 (mRFP1) and its variant mCherry, generated by mutagenesis, which are commonly used red fluorescent proteins. Marker genes coding for luminescence proteins may be exemplified by the luciferase (Luc) gene. A “selection gene” as referred to herein, is a gene encoding a protein which promotes selection of those transduced cells successfully expressing the vector form other cells. Such a so-called “selection protein” may be exemplified by a protein that confers resistance to one or more antibiotics, for example resistance to puromycin (Puro), Blasticidin (Bsd) and/or neomycin (Neo).

Non-limiting examples of multiple-promoter vectors include lentivector containing a fusion of different marker and selection genes such as Bsd-GFP, Bsd-RFP, Puro-EGFP, Puro-mCherry, Puro-Luc, EGFP-mCherry-Puro and the like.

The singular terms “a” “an” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia and is used herein to indicate a non-limiting example. Thus, the abbreviation “e.g.” is synonymous with the term “for example”. In case of conflict, the present specification, including explanations of terms, will control. In addition, all the materials, methods, and examples are illustrative and not intended to be limiting.

### III. Overview of Several Embodiments

The present disclosure is based on a discovery by the present inventors that certain miRNAs, either alone or in combination, play an important role in ALL progression via NAMPT regulation. The present inventors have, thus, envisaged that expression levels of one or more miRNA may be useful as biomarkers for the identification of patients who are most likely to benefit from treatment with NAMPT inhibitors.

The relevance of various miRNA expression levels on the growth rate of leukemic cells has been evaluated by the present inventors, as described in the Examples section herein, using in vitro cell line model and a xenograft mice model they have developed. For example, NAPMT, a key enzyme in the NAD⁺ pathway, was validated utilizing these models, as a novel target of miR-451 for direct binding thereto, and regulation thereof.

The present disclosure provides, in an aspect, the use of miR-451 as a sole biomarker in prognosis of ALL relapse risk and in diagnosing a patient that may benefit from a treatment modality featuring NAMPT inhibition.

Methods described herein include, for example:

(i) a method for prognosis of a treatment modality that affects a metabolic pathway associated with miR-451 expression in a patient afflicted with ALL, particularly but not exclusively, a treatment modality that inhibits NAMPT;

(ii) a method for identifying or diagnosing an ALL patient that can benefit from a treatment modality that affects a miR-451-related metabolic pathway, particularly but not exclusively, a treatment modality that inhibits NAMPT;

(iii) a method for early diagnosis of ALL relapse in a subject; and

(iv) a method for treatment of ALL in a patient that can benefit from a treatment modality that affects a metabolic pathway associated with miR-451 expression, particularly but not exclusively, a treatment modality that inhibits NAMPT.

Methods (i)-(iv) comprise the step of determining the level of expression of miR-451 in a biological sample obtained from the subject, and if the level of expression of miR-451 is below a predetermined level, correlating the level of expression of miR-451 with (i) a positive prognosis of a treatment modality that affects a metabolic pathway associated with miR-451 expression; (ii) compliance of the patient with a treatment modality that affects a miR-451-related metabolic pathway; (iii) ALL relapse risk; and/or (iv) a positive response of the patient to a treatment modality that affects a metabolic pathway associated with miR-451 expression, for example, positive response to NAMPT inhibition.

“Early diagnosis”, as referred to herein, is at the time of first diagnosis of the disease without the need to wait for initial treatment response. The ability to early diagnose relapse risk is critical as it enables to optimally direct or dictate an early therapeutic intervention and/or establish a treatment modality that would reduce relapse risk at later stages of the disease already upon first diagnosis thereof.

In exemplary embodiments, a metabolic pathway associated with miR-451 expression is NAMPT-regulated NADH biosynthesis, and in accordance with these embodiments, the effective treatment modality is inhibition of NAMPT.

In exemplary embodiments, NAMPT inhibition is effected by one or more inhibitors selected from the group consisting of a small molecule inhibitor, antibody, antisense nucleic acid, miRNA and RNA interference agent. For example, the NAMPT inhibitor may be a ribonucleic acid sequence at least 90% identical to a miR-451 ribonucleic acid sequence of SEQ ID NO: 2, or a nucleic acid expressing a ribonucleic acid sequence at least 90% identical to a miR-451 ribonucleic acid sequence of SEQ ID NO: 2, and any combination thereof. A NAMPT inhibitor may be further exemplified by the small molecule FK866 or a functional variant thereof.

The biological sample used for measuring miR-451 expression levels may be bone marrow, lymph fluid, whole blood, plasma, serum or central nervous system (CNS) fluid.

Any of the methods described herein are applicable to a pediatric ALL patient, for example, an adolescent, a child, or an infant inflicted with B-cell ALL.

The present disclosure provides, in another aspect, a pharmaceutical composition (also referred to herein simply as “composition”), for use in methods of treating treatment of ALL in a subject, for example, in preventing relapse of ALL after remission of the disease, or in reducing the risk of relapse in a subject. The described compositions may be administered to the subject, thereby treating the ALL.

In some embodiments, a described pharmaceutical composition comprises an inhibitor of NAMPT, and the composition is used for treatment of ALL in a subject that has been diagnosed as presenting low levels of miR-451.

In some embodiments, the inhibitor of NAMPT is selected from the group consisting of a small molecule inhibitor, antibody, antisense nucleic acid, miRNA and RNA interference agent. In exemplary embodiments, the inhibitor is FK866 or a functional variant thereof.

In exemplary embodiments, the inhibitor of NAMPT is miR-451, and a described pharmaceutical composition for treatment of ALL may comprise a ribonucleic acid sequence at least 90% identical to a miR-451 ribonucleic acid sequence set forth as SEQ ID NO: 2, or a nucleic acid expressing a ribonucleic acid sequence at least 90% identical to a miR-451 ribonucleic acid sequence set forth as SEQ ID NO: 2. In a particular embodiment, the nucleic acid expressing miR-451 is operably linked to a recombinant expression plasmid.

In some embodiments, a described pharmaceutical composition for use in a method of treatment of ALL in a subject comprises an inhibitor of miR-1290. In exemplary embodiments, the inhibitor of miR-1290 includes a nucleic acid that is at least 90% identical to the reverse complement of the miR-1290 sequence as set forth in SEQ ID NO:3. In other embodiments, the inhibitor of miR-1290 includes a nucleic acid expressing a nucleic acid that is at least 90% identical to the reverse complement of the miR-1290 sequence as set forth in SEQ ID NO:3.

In particular embodiments, the inhibitor of miR-1290 is selected from the group consisting of a DNA inhibitor or an RNA interference (RNAi) agent.

In some embodiments, a described pharmaceutical composition for treatment of ALL comprises a ribonucleic acid sequence at least 90% identical to a miR-451 ribonucleic acid sequence set forth as SEQ ID NO:2, or a nucleic acid expressing a ribonucleic acid sequence at least 90% identical to a miR-451 ribonucleic acid sequence set forth as SEQ ID NO:2, and further comprises an inhibitor of miR-1290 comprising a nucleic acid expressing a nucleic acid that is at least 90% identical to the reverse complement of the miR-1290 sequence as set forth in SEQ ID NO:3.

In some embodiments, a described pharmaceutical composition for treatment of ALL comprises an inhibitor of Janus kinase 2 (JAK2). Particular examples of the JAK2 inhibitor include a small molecule inhibitor, antibody, antisense nucleic acid, and RNA interference agent.

The present disclosure provides, in yet another aspect, methods for treatment of a subject with an increased risk of ALL relapse that include first determining the expression level of miR-1290 and at least one of miR-151-5p and miR-451; and comparing the determined expression of miR-1290, and miR-151-5p and/or miR-451 with control expression of miR-1290, and miR-151-5p and/or miR-451, wherein a significant increase in miR-1290 expression in the subject in comparison to the control miR-1290 expression, combined with a significant decrease in expression of the at least one of miR-151-5p and miR-451 in comparison to the control expression of miR-151-5p and/or miR-451, indicates that the subject has an increased risk of ALL relapse, and requires treatment appropriate for a subject with an increased risk of ALL relapse; and then administering to the subject a therapeutically effective amount of a composition comprising an inhibitor of NAMPT, or any of the other compounds or compositions described herein for use in treating ALL.

In some embodiments, a method for treatment of pediatric ALL in a subject, comprises the steps of administering to the subject a therapeutically effective amount of an inhibitor of NAMPT, selected from the group consisting of an antibody, antisense nucleic acid, microRNA (miRNA) and RNA interference agent, thereby treating the patient, with the proviso that the inhibitor of NAMPT is not miR-451.

### IV. ALL Prognosis by Detection of miR-1290, miR-151-5p, and miR-451

Prediction of relapse has proved to be the key for successful treatment of pediatric ALL. Described herein is the observation that even on the day of first ALL diagnosis, differences in miRNA expression are predictive of disease relapse, and indicative of the appropriate form of treatment to provide a patient. For example, described herein is the observation that overexpression of miR-1290 correlates with ALL relapse, and the predictive power of combination determinations of miR-151 and miR-451 expression (underexpressed, compared with a standard), and miR-1290 expression (overexpressed, compared with a standard) is greater than any subcombination thereof (e.g., determinations of miR-1290 and miR-151, or determinations of miR-1290 and miR-451).

Current practice for ALL treatment includes determining the risk of disease relapse following standard treatment. The determined risk prognosis is determinative of the treatments given to the patient. Standard prognosis methods for determining risk include, for example, the Children Oncology Group (COG), Berlin-Frankfurt-Münster (BFM), minimal residual disease (MRD), United Kingdom ALL (UKALL) group, Chinese Children's Leukemia Group (CCLG), and Dana-Farber Cancer Institute (DFCI) ALL Consortium systems, from which a patient is determined to be high risk (HR), intermediate risk (IR), and standard risk (SR). Accordingly, under current practice, ALL treatment is provided as a risk-based treatment, i.e., high risk patients receive a more intensive treatment while the standard risk patients receive treatment reduction.

According to the BFM system, e.g., BFM-2000, (Vrooman et al., Curr Opin Pediatr. (2009); 21:1-8), standard risk includes (1) no adverse cytogenetic abnormalities; (2) age between 1 and 6 years; and (3) good response to prednisone treatment on day 8. High risk includes at least one of (1) cytogenetic abnormalities (e.g. t(9;22) and t(4;11)) translocations); (2) under 1 year of age or above 6 years; (3) poor response to prednisone treatment on day 8; and (4) hypodiploidy. Intermediate risk includes those whose age is between 1 to 6, show no adverse cytogenetic abnormalities, no hypodiploidy and a good response to prednisone on day 8 of treatment, as well as those whose condition does not meet the criteria for either standard risk or high risk.

An alternative definition of relapse risk is MRD diagnosis, which is based on an indication of the amount of remaining leukemic blasts in a patient's bone marrow (BM) during and/or after treatment, which can be measured by means of flow cytometry (FACS) and polymerase-chain reaction (PCR) (van Dongen et al., Lancet. (1998); 352:1731-1738). MRD risk stratification is performed after MRD analysis on days 33 and 78 from the beginning of treatment. MRD standard risk is defined as a negative MRD finding on day 33. MRD high risk is defined as a finding of 10⁻³ leukemic cells (1 leukemic cell in 1000 normal cells) on day 78. All other findings are defined as intermediate risk. In the present disclosure, the MRD test was performed by PCR amplification of immunoglobulin and T-cell receptor gene rearrangement sites (PCR-MRD) and interpreted according to the guidelines of the European Study Group for MRD detection in ALL (ESG-MRD-ALL).

Prognostic grouping by BFM-2000 clinical risk grouping does not normally dictate a different treatment regime for the diagnosed patient. However, once MRD risk classification becomes available after day 78 of treatment, it replaces the previous classification and provides a basis for planning treatment for the patient. Until such time that the MRD risk group prognosis replaces the previous risk classification, a standard treatment is provided to all patients.

According to the COG system (Smith et al., J. Clin Oncol. (1996); 14:18-24; Hunger, Am Soc Clin Oncol Educ Book (2012); 611-615), National Cancer Institute (NCI) standard risk includes (1) WBC count less than 50,000/UL; and (2) age 1 to younger than 10 years. NCI high risk includes (1) WBC count 50,000/UL or greater; and/or (2) age 10 years or older.

In addition to the time-honored features of age and WBC, the presence of extramedullary disease (central nervous system (CNS) or overt testicular involvement) is a factor used to determine the intensity of treatment. CNS disease is defined as more than five white blood cells per ml of spinal fluid (in a nonbloody sample) which are blasts morphologically (referred to as “CNS3”). There is an intermediate state, “CNS2,” in which there are fewer than five cells per ml but blasts are detectable by cytocentrifugation, a procedure that concentrates the leukemic cells and increases diagnostic sensitivity. In “CNS1,” there is no evidence of CNS involvement (fewer than five cells per mm³ and no blasts).

Induction drugs are given at first four weeks of treatment. The goal of induction treatment, also referred to as remission induction therapy, is to clear the blood and bone marrow of blasts and bring about a complete remission, or complete response. NCI standard risk without CNS3 or overt testicular disease induction drugs include (1) dexamethasone; (2) vincristine; and (3) asparaginase. NCI high risk with CNS3 or overt testicular disease induction drugs include (1) dexamethasone; (2) vincristine; (3) asparaginase; and (4) an anthracycline such as daunorubicin (Borowitz et al., Blood (2008); 111:5477-5485).

The described methods therefore not only allow for improved determination of ALL prognosis and relapse risk, but also improved overall systems of treatment for ALL, which include providing the most appropriate treatment protocol as determined by the determined relapse risk at a significantly earlier time point than currently achievable with MRD testing.

Accordingly, provided herein are methods for the prognosis of ALL in a subject, by determining the level of expression of miR-1290, alone or in combination with the expression of miR-151-5p and/or miR-451, and comparing the determined expression to a control or standard, such as a predetermined cut-off value. In a particular embodiment, the expression of miR-1290 is detected. In another embodiment, the expression of miR-1290 and miR-151-5p is detected. In yet another embodiment, the expression of miR-1290 and miR-451 is detected.

In the described methods, the expression in the subject sample of miR-1290, alone or in combination with the expression of miR-151-5p and/or miR-451 is compared to the expression of the specific miRNAs in a control sample, wherein a comparative significant increase in miR-1290 expression alone or in combination with a significant decrease in at least one of miR-151-5p and miR-451 indicates an increased risk of relapse. As understood herein, a control is a standard defined by the amount of specific miRNA expression in samples from one of more subjects who are, for example, either ALL-free or, alternatively, who had ALL but did not relapse. Such standards can change over time as additional patient data is accumulated.

In some embodiments, the predetermined control value to which a subject sample is compared, is described as a cut-off value, wherein a departure from the cut-off indicates a significant difference from the control value, and an increased risk of ALL relapse. In such embodiments, the expression of the miRNAs in relation to the cut-off value determines how the patient should be grouped with those pre-established ALL patient populations associated with specific relapse rates. For example, determination that a patient is expressing miR-1290 at levels greater than a cut-off, combined with determination that at least one of miR-151-5p and miR-451 are expressed lower than a cut-off indicates that the patient has higher risk for relapse than a patient that does not exhibit such miRNA expression levels. As used herein, such expression (a detected downregulation of miR-151-5p and/or miR-451, and a detected upregulation of miR-1290) can be termed a “positive expression value”.

As described herein, a “cut-off value”, sometimes referred to as a “cut-off”, is a value that meets the requirements for both high diagnostic sensitivity (true positive rate) and high diagnostic specificity (true negative rate). Determined cut-off values are the result of a statistical analysis of miRNA expression value differences in pre-established populations which, e.g., either relapsed or remained disease-free.

A test yielding numeric values such as miRNA levels is referred to as a continuous test, and when the response of a diagnostic test is continuous, sensitivity and specificity can be computed across all possible threshold values. Sensitivity is inversely related with specificity in the sense that sensitivity increases as specificity decreases across various threshold. The receiver operating characteristic (ROC) curve is the plot that displays the full picture of trade-off between the sensitivity and (1-specificity) across a series of cut-off points. In a ROC curve, the true positive rate (sensitivity) is plotted in function of the false positive rate (1-specificity) for different cut-off points of a parameter. Each point on the ROC curve represents a sensitivity/specificity pair corresponding to a particular decision threshold. The area under the ROC curve (AUC) is a measure of how well a parameter can distinguish between two diagnostic groups (diseased/normal), and is considered as an effective measure of inherent validity of a diagnostic test. This curve is useful in finding optimal cut-off point to least misclassify, e.g., diseased or non-diseased subjects, and is one exemplary means to determine set of cut off values, herein embraced by the term “predetermined values”, “predetermined control values”, “predetermined expression levels” or “predetermined levels”, for example, predetermined miR-451 levels.

It should be emphasized that the accumulation of further patient data may improve the accuracy of the presently provided cut-off values, which are based on an ROC curve generated according to said patient data using, for example, a commercially available analytical software program. The miR-151-5p and/or miR-451 expression values are selected along the ROC curve for optimal combination of prognostic sensitivity and prognostic specificity which are as close to 100% as possible, and the resulting values are used as the cut-off values that distinguish between patients who will relapse at a certain rate, and those who will not (with said given sensitivity and specificity). The ROC curve may evolve as more and more patient-relapse data and related miR-151-5p, miR-451, and miR-1290 expression values are recorded and taken into consideration, modifying the optimal cut-off values and improving sensitivity and specificity. Thus, the provided cut-off values for miR-151-5p and/or miR-451 should be viewed as not limiting, but merely illustrative of the statistical analysis.

In an exemplary embodiment, the cut-off values for miR-151-5p and miR-451, respectively, are 0.00015 and 0.001 (units relative to expression of an internal standard; the determination of which is described in International Publication No. 2012/042516). Accordingly, respective miR-151-5p and miR-451 expression levels that are lower than 0.00015 and 0.001 indicate that a subject is expressing these miRNAs at lower levels than a control or expressing these levels below a predetermined level. With regard to miR-1290, if a subject is determined to be expressing miR-1290 above a determined cut-off value, the subject is identified as expressing miR-1290 at significantly higher levels than a control or expressing miR-1290 at significantly higher levels than a predetermined level.

In some embodiments, the determination of the miR-1290 expression combined with determination at least one of miR-151-5p and miR-451 is correlated with particular risks of relapse, depending on the determined expression levels. In other embodiments, the determined miRNA expression is combined with other clinical features, including white blood cell count (WBC), age, minimal residual disease (MRD) risk index, cytogenetic aberrations, response to prednisone treatment on day 8, and ploidity to determine disease prognosis and relapse risk.

In some embodiments, the ALL patient population group that may be examined by a described method is optionally further defined by sub-grouping of a patient according to at least one clinical criterion, and each patient sub-group belongs to a specific pre-established ALL patient population associated with a specific relapse rate. According to certain embodiments, the clinical criteria comprise subgroupings according to: B-cell ALL and/or T-cell ALL diagnosis; MRD high and low risk definitions; response to prednisone on day 8 of treatment; BFM high and low risk definitions; WBC being over or below 20,000 cells/ml; patient age being over one and under six years old or otherwise; children's cancer group (CCG) high and low risk definitions; and gender.

Typically, a good response to prednisone on day 8 of treatment is defined as a finding of less than 1000 leukemic blast cells/ml of blood, whereas a poor response is defined as a finding of more than 1000 leukemic blast cells/ml of blood.

In particular embodiments, the described method is specifically applicable for predicting B-cell ALL relapse.

The miRNAs described herein can be detected by any methods known to the art, including use of standard oligonucleotides primers and probes, each of which can specifically hybridize to a nucleic acid sequence of at least one of miR-151-5p (SEQ ID NO:1), miR-451 (SEQ ID NO:2), and miR-1290 (SEQ ID NO:3), and of at least one control reference miRNA.

Such sequences include sequences that are 100% identical to the reverse complement of SEQ ID NOs:1-3. It is understood that such primers and probes can also be less than identical to the reverse complement of SEQ ID NOs:1-3, such as 98%, 95%, 90%, 85% or even less, and that the design of such primers is well known in the art.

Non-limiting examples of standard nucleic acid detection methods include PCR (in all of its forms, including quantitative PCR), nucleic acid microarrays, Northern blot analysis, and various forms of primer extension.

Primers and probes for use in detecting the described miRNAs can be RNA or DNA, or analogs thereof. Examples of DNA/RNA analogs include, but are not limited to, modifications such as 2-′O-alkyl sugar modifications, methylphosphonate, phosphorothiate, phosphorodithioate, formacetal, 3-thioformacetal, sulfone, sulfamate, and nitroxide backbone modifications. Analogs are further exemplified by locked nucleic acids (LNA) analogs, wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs. Probes may also be mixtures of any of the oligonucleotide analog types together or in combination with native DNA or RNA. In particular embodiments, the oligonucleotides and analogs can be used alone; in other embodiments, they can be used in combination with one or more additional oligonucleotides or analogs.

In a particular embodiment, the described oligonucleotides are any one of a pair of primers or nucleotide probe, for use in detecting the level of expression of miR-1290, miR-151-5p and/or miR-451, using a nucleic acid amplification and/or detection assay including but not limited to PCR, quantitative reverse transcription PCR (RT-qPCR), micro arrays, in situ hybridization and Comparative Genomic Hybridization. Methods and hybridization assays using self-quenching fluorescence probes with and/or without internal controls for detection of nucleic acid application products are known in the art, for example, U.S. Pat. Nos. 6,258,569; 6,030,787; 5,952,202; 5,876,930; 5,866,336; 5,736,333; 5,723,591; 5,691,146; and 5,538,848.

In particular embodiments, in addition to detection of the miRNA of interest (miR-451, miR-1290, and the like), the particular detection methods also utilize primers and/or probes to detect the expression of a nucleic acid to be used as an internal normalizing control. According to these embodiments, the detecting nucleic acid molecules used by the described methods include isolated oligonucleotides (e.g., probes or primers) that specifically hybridize to a nucleic acid sequence of miR-1290, miR-151-5p and/or miR-451 and, in addition, isolated oligonucleotides (e.g., probes or primers) that specifically hybridize to a nucleic acid sequence of at least one reference RNA. Non-limiting examples of such reference RNAs include a reference miRNA (whose expression is known to be the same, regardless of ALL condition), the 5S ribosomal RNA (rRNA), or the U6 small nuclear RNA. Desired isolated oligonucleotides may be obtained, for example, form a Universal Reference (UR) pool such as the miRXplore™ UR (Miltenyi Biotech), which represents a pool of over 1000 synthetic miRNA.

The described methods relate to prognosis of ALL based on examining the expression of certain miRNA's, specifically, miR-151-5p, miR-451, and miR-1290 in a test sample, specifically, a biological sample obtained from a subject, methods of processing such samples to isolate nucleic acids for use in the described methods are known to the art. In particular embodiments, the sample is derived from the bone marrow of the subject.

### V. Compositions for ALL Treatment and Methods of Use Thereof

Described herein are the observations that miR-451 and miR-151-5p expressions are significantly decreased in ALL subjects with a greater risk of ALL relapse. Conversely, it was also observed that miR-1290 expression is significantly increased in ALL subjects with a greater risk of ALL relapse. Additionally, described herein are specific targets of miR-451 and miR-1290 translational regulation. From these observations, compositions, and methods of their use for ALL treatment (including inhibiting ALL relapse) are accordingly indicated as detailed below.

miR-451 and miR-151-5p Expression

Described herein are compositions for use in methods of treating ALL, including decreasing the risk for ALL relapse, by increasing the levels of miR-151-5p and/or miR-451 in a subject. The described methods include administering to a subject in need thereof a therapeutically effective amount of at least one of miR-151-5p and miR-451, or any nucleic acid sequence encoding at least one of miR-151-5p and miR-451, or the pri-miRNA or pre-miRNA thereof. Administration of the miR-151-5p and/or miR-451 increases the intracellular level of the miRNA in a treated subject by 10%, 15%, 20%, 25%, 30%, 40%, 50% or even more than in a non-treated subject, thereby treating the ALL and/or inhibiting its relapse. In particular embodiments, the miRNA is administered to the subject. In other embodiments, the administered nucleic acid sequence includes an expression vector or engineered plasmid encoding at least one of miR-151-5p and miR-451.

The miR-151-5p and/or miR-451 sequences to be administered (whether directly or in a miRNA-encoding vector such as a lentivector) are set forth as SEQ ID NOs:1 and 2, respectively. Preferably, a human sourced miRNA (hsa-miRNA) is employed, for example, synthetic hsa-miR-451 and hsa-miR-151-p. Functional variants of these sequences can also be used in the described methods, as long as the specific translation regulation function of miR-151-5p and miR-451 is retained. Such sequence variants can be 98%, 95%, 90%, 85%, or even less identical to the wild type miRNA sequences set forth as SEQ ID NOs:1 and 2.

Inhibition of miR-1290 Expression

Additionally, described herein are compositions for use in methods of treating ALL, including inhibiting ALL relapse, by decreasing the levels of miR-1290 in a subject and/or inhibiting miR-1290 translational regulatory activity. The described methods include administering to a subject an inhibitor of miR-1290 expression, e.g., formulated as a pharmaceutical composition. Administration of the miR-1290 inhibitor (or nucleic acid encoding such inhibitors) decreases the intracellular level of the miRNA in a treated subject by 10%, 15%, 20%, 25%, 30%, 40%, 50% or even more than in a non-treated subject, thereby treating the ALL and/or inhibiting its relapse. In some embodiments, the inhibitor is a nucleic acid molecule capable of specifically hybridizing to miR-1290, such as a nucleic acid comprising the reverse complement of the miR-1290 sequence set forth herein as SEQ ID NO:3. Such nucleic acids include DNA and RNA antisense inhibitors as known in the art and as described herein. Particular non-limiting examples of miR-1290 inhibitors include DNA antisense oligonucleotides, morpholino oligonucleotides, and RNA interference agents such as siRNA which target the miR-1290 sequence. It will be appreciated that the antisense inhibitor or targeting agent of miR-1290 need not contain a reverse complementary sequence that is 100% complementary to the miR-1290 sequence. Antisense sequence variants can be used in the described methods. Such sequence variants can be 98%, 95%, 90%, 85%, or even less identical to the reverse complementary sequence set forth as SEQ ID NO:3.

A wide variety of methods for delivering a nucleic acid to a subject are known in the art. Such methods are contemplated equally for administration of nucleic acids whether for use in increasing expression of miR-151-5p and miR-451 or use in inhibiting expression of miR-1290.

In some embodiments, a miRNA-encoding nucleic acid or nucleic acid encoding a miRNA inhibitor is operably linked to a recombinant plasmid that is operable in a mammalian cell. In other embodiments, the miRNA-encoding nucleic acid or miRNA inhibitor (or nucleic acid encoding the inhibitor) is incorporated into a viral vector, such as a lentivirus vector.

In some embodiments, the provided miRNA or miRNA inhibitor, e.g., antisense inhibitor, is not provided in a vector or plasmid but is provided in a form for immediate use (e.g., the miRNA or inhibitor is not encoded by another nucleic acid that needs to be transcribed to carry out its function as a miRNA or miRNA antisense inhibitor). In such embodiments, the miRNA or miRNA inhibitor is provided in any composition that provides stability to nucleic acid and/or facilitates the uptake of the nucleic acid to a cell. Such delivery compositions are well known in the art, and include but are not limited to, liposomes, micelles (and inverted micelles), micro- and nano-particles of nucleic acid complexed with a degradable polymer, degradable nucleic acid-polymeric implants, exosomes, e.g., natural or engineered exosomes (exosomes are specialized membranous nano-sized vesicles derived from endocytic compartments in many cell types) and the like.

### Inhibitors of NAMPT or JAK2

Nicotinamide phosphoribosyl transferase (NAMPT) regulates NAD⁺ synthesis, and by extension apoptosis. Various malignancies overexpress NAMPT including colorectal, ovarian, breast, gastric, prostrate, carcinomas, myeloma, melanoma, leukemia and lymphomas. Higher NAMPT expression is associated with a poor prognosis and increased tumor growth, metastasis and dedifferentiation. NAMPT levels distinguish between benign and malignant tissue as well as correlate with a more aggressive malignant phenotype.

Described herein is the observation that NAMPT is negatively regulated by miR-451. In the present disclosure, the direct binding of miR-451 to its target, NAMPT is identified. High expression of miR-451 resulted in the decreased NAMPT protein level, while miR-451 silencing resulted in elevated levels of the NAMPT protein. Thus, miR-451 can regulate metabolic pathways that are NAMPT dependent. Such metabolic pathways are also termed herein “metabolic pathways associated with miR-451 expression”.

The aggressiveness of NAMPT upregulating cells suggest that miR-451 may be a key regulator of several metabolic pathways controlling cell aggressiveness in cancer and a possible candidate in the mechanism leading ALL cells to become increasingly aggressive.

Similarly, it was discovered that SOCS4, an inhibitor of the cancer-promoting gene JAK2 (Janus Kinase 2) is negatively regulated by miR-1290. Accordingly, compositions and methods of their use are described herein for treating ALL, including inhibiting ALL relapse. The methods include administering to a subject in need thereof a therapeutically effective amount of an inhibitor or antagonist of NAMPT or and/or an inhibitor or antagonist of JAK2.

Non-limiting examples of the antagonists and inhibitors of NAMPT for use in the described uses, compositions, and methods include: anti-NAMPT antibodies or fragments thereof which are able to bind NAMPT; small molecule agents, such as but not limited to, FK866, also known as WK175 or AP0866 (Sigma), CHS828 also known as GMXI778 or GMXI777, and EB1627 also known as Teglarinad (Galli et al. 2013, J. of Medical Chemistry 56:6279-6296), which interact with NAMPT and interfere with its biological function; NAMPT competing derivatives (peptide and non-peptide based); antisense oligonucleotides; a nucleic acid which is capable of hybridizing with at least part of a gene encoding NAMPT and inhibit its expression, such as siRNA and miRNA; ribozymes; and molecules that target NAMPT promoter transcription factors or bind to the NAMPT promoter, thereby blocking access to such transcription factors and preventing its expression.

In some embodiments, the miRNA inhibitor of NAMPT expression is miR-451.

Likewise, non-limiting examples of antagonists and inhibitors of JAK2 for use in the described uses, compositions, and methods include: anti-JAK2 antibodies or fragments thereof which are able to bind JAK2; small molecule agents, which interact with JAK2 and interfere with its biological function; JAK2 competing derivatives (peptide and non-peptide based); antisense oligonucleotides; a nucleic acid which is capable of hybridizing with at least part of a gene encoding JAK2, and inhibit its expression, such as siRNA and miRNA; ribozymes; and molecules that target JAK2 promoter transcription factors or bind to the JAK2 promoter, thereby blocking access to such transcription factors and preventing its expression.

In particular embodiments, the NAMPT antagonist is a small molecule antagonist that interacts with NAMPT and inhibits NAD⁺ synthesis. A non-limiting example of a small molecule for use in the current disclosure is FK866, which has a structure:

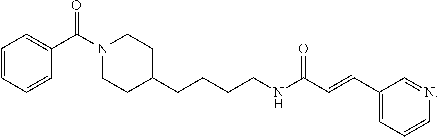 [SMILES: O=C(/C=C/c1cccnc1)NCCCCC1CCN(C(=O)c2ccccc2)CC1]

FK866 is a highly specific, non-competitive NAMPT inhibitor that induces a gradual NAD⁺ depletion, ATP depletion, and delayed cell death by apoptosis. Based on its promising preclinical activity, FK866 was proposed as a novel drug to combat different malignancies. FK866 was well tolerated and safe when tested in a Phase I study in patients with advanced cancers. Functionally equivalent variants and derivatives of FK866 are also contemplated herein.

In other embodiments, the antagonist for use in the described methods is an anti-NAMPT or anti-JAK2 antibody or fragments thereof which are able to recognize and bind NAMPT or JAK2. Antibodies that specifically recognize NAMPT or JAK2 would recognize and bind the particular protein (and peptides derived therefrom) and would not substantially recognize or bind to other proteins or peptides found in a biological sample. The determination that an antibody specifically detects its target protein is made by any one of a number of standard immunoassay methods; for instance, the Western blotting technique (Sambrook et al., In Molecular Cloning: A Laboratory Manual, CSHL, New York, 1989).

Also contemplated are humanized antibodies, for instance humanized equivalents of a murine monoclonal antibodies. A “humanized” immunoglobulin is an immunoglobulin including a human framework region and one or more complementarity-determining regions (CDRs) from a non-human (for example a mouse, rat, or synthetic) immunoglobulin. The non-human immunoglobulin providing the CDRs is termed a “donor”, and the human immunoglobulin providing the framework is termed an “acceptor”. In one embodiment, all the CDRs are from the donor immunoglobulin in a humanized immunoglobulin. Constant regions need not be present, but if they are, they must be substantially identical to human immunoglobulin constant regions, such as at least about 85-90%, or such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering as well known in the art.

In still further embodiments, the NAMPT or JAK2 inhibitor is an inhibitor of NAMPT or JAK2 gene expression. NAMPT or JAK2 expression can be inhibited or eliminated at the level of transcription or at the level of translation. In particular examples, NAMPT or JAK2 expression is inhibited by use of antisense oligonucleotides, antisense morpholinos oligonucleotides, or any other nucleic acid which is capable of hybridizing with at least part of a gene encoding NAMPT or JAK2 (or the RNA product thereof), and inhibiting its expression Such nucleic acids include, for example, siRNA, shRNA, and miRNA.

Suppression of endogenous NAMPT or JAK2 expression can also be achieved using ribozymes. Ribozymes are synthetic molecules that possess highly specific endoribonuclease activity. The production and use of ribozymes are disclosed, for example, in U.S. Pat. Nos. 4,987,071 and 5,543,508. The inclusion of ribozyme sequences within antisense RNAs may be used to confer RNA cleaving activity on the antisense RNA, such that endogenous mRNA molecules that bind to the antisense RNA are cleaved, which in turn leads to an enhanced antisense inhibition of endogenous gene expression. Suppression can also be achieved using RNA interference, using known and previously disclosed methods as described herein.

It will be appreciated that when administered to a subject, the compositions for use in the described treatment methods, including NAMPT or JAK2 inhibitory and antagonist compounds, and the miR-451, miR-151-5p, and miR-1290-inhibitory nucleic acids described herein, are formulated in standard formulations known in the art for pharmaceutical compositions. Such compositions can further include any standard pharmaceutically acceptable salts, carriers, and excipients know in the art which are appropriate for any particular mode of administration.

### Combination Therapies

In particular embodiments, the compositions for use in treating ALL can be administered in varying combinations and with additional therapeutic agents, such as one or more immunomodulatory agents or known cancer therapeutic, such as a chemotherapeutic agent of the anthracycline family used when a subject is determined to have an increased risk of relapse. For example, in particular embodiments, miR-451 can be co-administered with an inhibitor of miR-1290. Similarly, in particular embodiments, a NAMPT antagonist, such as FK866 is co-administered with an inhibitor of miR-1290, such as an antisense DNA oligonucleotide containing the reverse complement of SEQ ID NO:3, and/or with miR-451.

When administered as part of a combination treatment, each composition can be administered separately in an additional separate step having an optional different mode of administration, or together in a single pharmaceutical combination.

### VI. Methods for ALL Treatment

Additionally, described herein are methods (also sometimes referred to herein as “systems”) for treating an ALL patient.

In some embodiments, a described method involves first determining the risk of ALL relapse, through the described methods of detecting the expression of miR-1290, and at least one of miR-151-5p and miR-451, as combined biomarkers.

In some embodiments, a described method involves first determining the risk of ALL relapse, by a described diagnosis or prognosis method based on solely detecting miR-451 expression, as a sole biomarker.

Once ALL relapse risk is determined (e.g. once a subject is grouped as standard risk, intermediate risk, or high risk), an appropriate treatment is given, tailored to the determined relapse risk, and tailored to the molecule, e.g., miRNA determined to be deficient or overexpressed.

Prior to the described methods, determining of an appropriate ALL treatment not only relied on entirely different clinical parameters (e.g. WBC count, prednisone response), but such determinations were made days or even weeks after the initial diagnosis. In contrast, the disclosed methods can determine appropriate treatment at the time of initial or first ALL diagnosis, following a test for expression of miR-1290, and at least one of miR-151-5p and miR-451, or alternatively, following a test for expression of miR-451 alone. For example, if miR-451 is determined to be significantly underexpressed, the described methods may include administering an antagonist of NAMPT, such as FK866, or similarly administering miR-451 to the subject so as to inhibit NAMPT expression.

The current methods (systems) are based on the understanding that modern healthcare services are provided by large entities within which multiple healthcare services are given to a patient. Particular non-limiting examples of such entities include physicians' groups, hospital consortiums or networks, and public or private health maintenance organizations. Within these entities, a patient's health care may be managed by a single actor, such as a physician, nurse practitioner, and the like, but specialized services are provided to the patient by multiple actors within the system, such as diagnosticians and specialists. It is recognized that in particular embodiments, certain services may be outsourced to a provider outside of the main service provider. However, in all embodiments, it is the main service provider, or representative or employee thereof, who is directing the described systems of treatment.

### VII. miR-451 as Tumor Suppressor and as a Unique Biomarker for Early Diagnosis of ALL Relapse Risk

Several studies have indicated that miR-451, which is part of the ALL miRNA panel that predicts, according the present disclosure, relapse at the time of diagnosis, acts as a tumor suppressor gene in several cancers including leukemia. miR-451 has been shown to be downregulated in pediatric ALL compared with healthy samples (Ju X et al., Pediatr Hematol Oncol (2009) 26(1):1-10; de Oliveira et al., Pediatr Blood Cancer (2012) 59(4):599-604).

Described herein is the observation that miR-451 may be a stand-alone diagnostic marker of relapse risk. It has been established herein, in a retrospective study, that miR-451 was significantly decreased in patients who relapsed versus patients in long-term remission, and that low expression of miR-451 could independently predict relapse with a hazard ratio of 11.3 in the ALL cohort (p=0.001).

Disclosed herein are methods for diagnosis of ALL risk relapse which are based on miR-451 expression levels in a sample obtained from a subject, e.g., an ALL patient. The miR-451 expression level in the sample is compared to the expression of miR-451 in a control sample, wherein a comparative significant decrease in miR-451 indicates an increased risk of relapse. The control miR-451 level may be a predetermined level as described herein. For example, determination that a patient is expressing miR-451 at levels lower than a cut-off indicates that the patient has higher risk for relapse than a patient that does not exhibit such miRNA expression levels.

In some embodiments, the determination of the miR-451 expression is correlated with particular risks of relapse, depending on the determined expression levels. In other embodiments, the determined miRNA-451 expression is combined with other clinical features, including white WBC, age, minimal residual disease (MRD) risk index, cytogenetic aberrations, response to prednisone treatment on day 8, and ploidity to determine disease prognosis and relapse risk.

Described herein is the observation that miR-451 may be a tumor suppressor. For example, in in vivo experiments in a mice xenograft ALL model described herein, growth rate of tumors overexpressing miR-451 was significantly reduced compared to miR-451 silenced cells (p=0.03).

### VIII. miR-451 Expression Levels as a Biomarker for Assessing Compliance with a NAMPT-Inhibition Treatment Modality

Tumor cells are characterized by increased NADH and ATP catabolism, rendering them more sensitive to NAMPT inhibition than benign cells. Attempts to identify biomarkers for patients selection for FK866 treatment lead to several promising directions. Several groups demonstrated an inverse correlation between NAMPT expression levels and sensitivity to its inhibitors. Described herein is the observation that mice expressing low levels of miR-451, correlating with increased NAMPT expression, demonstrated significantly increased sensitivity to treatment with a NAMPT inhibitor in a xenograft ALL model (p=0.0001). In exemplary embodiments described herein, mice injected with ALL cells expressing low miR-451 levels, were significantly more sensitive to FK866 treatment. These mice expressed low miR-451 levels which correlated with upregulation of NAMPT protein levels and significantly elevated tumor growth. Thus, a role has been established herein for miR-451 as a biomarker in detecting the sensitivity to, or compliance with, specific NAPMT inhibition treatment modalities in ALL treatment.

ALL cells are highly dependent on the miR-451-NAMPT pathway and warrant the potential use of miR-451 expression levels as a novel biomarker in the selection of a sub-group of ALL patients who may benefit from treatment with NAMPT inhibitors.

The following examples are provided to illustrate certain particular features and/or embodiments. These examples should not be construed to limit the disclosure to the particular features or embodiments described.

### EXAMPLES

### Materials and Methods

### Patient Material Collection

Bone marrow (BM) biopsies at the time of diagnosis were obtained from 95 pediatric ALL patients and the samples elected for further studies were those with at least 80% leukemic blasts. Fifty-three (53) males and 42 females, median age 6.6 years (range 0.3-18); 28 patients had T-cell ALL, 46 patients had a WBC >20000, 18 patients were poor prednisone responders; 32 patients were clinically classified as BFM high-risk, 40 intermediate-risk and 23 standard-risk; 31 patients relapsed. The median follow-up of patients was 69 months (range 6-296). All patients were treated at Schneider children's medical center of Israel. Four (4), 16, 25 and 50 patients were treated according to the INS-84, INS-89, INS-98 and INS-2003 protocols respectively (Stark B et al., Leukemia. (2010); 24: 419-424; INS-2003).

### RNA Extraction

Total RNA was isolated from 10⁷ cells from BM biopsies (e.g., BM aspirates) using RNeasy® Mini kit (Qiagen, Hilden, Germany) according to the manufacturer's instruction. RNA concentration was determined by measuring the absorbance at 260 nm with a A260/A180 ratio of 1.8 (The wavelength of maximum absorption for both DNA and RNA is 260 nm (λmax=260 nm) with a characteristic value for each base. The ratio of absorbance at 260 nm and 280 nm, A260/A280, is used as a means to estimate purity of RNA preparations obtained from biological samples and assess contamination by proteins. Pure RNA preparations have an A260/A280 ratio of greater than or equal to 1.8).

miRNAs Expression Profile

Measuring the changes in the miRNAs expression profile is extremely important for deciphering the biological context of differentially expressed genes. In miRNA profiling, as used herein, a specific miRNAs pattern called a profile is obtained from miRNA isolated from a biological sample, for example, bone marrow, blood or CNS fluids of a subject inflicted or suspected of being inflicted with ALL. Optionally, miRNAs expression profiling is performed by the use of a multiplex molecular means, namely, a means which affords the handling of multiple or multitude molecules simultaneously in a single assay or in a single experiment, wherein “multiplex” and “multiple”, as used herein, denote at least two, for example, two, three, four, five and even more. Non-limiting examples of multiplex molecular means useful for miRNA profiling include microarrays and multiplex RT-qPCR.

In exemplary embodiments, miRNA expression profiles were conducted using a microarray. Generally, microarray is a collection of multiple microscopic spots also termed “features” or “tests spots” on a solid support, each feature accommodating or comprising one or more copies of specific, single-stranded DNA or RNA termed herein “probes”, which can be probed with complementary single-stranded (matching) target DNA and/or RNA obtained from a biological sample. For example, in a miRNA microarray, single antisense strands of miRNA anchored onto a solid support are allowed to hybridize to complementary sequences of sample target miRNAs. The term “complementary” as used herein means that the sequence of the probe is exactly hybridizing to the sequence of the target. In general terms, probes are synthesized and immobilized onto discrete features, or test spots. Each feature may contain millions of identical probes. The target may optionally be fluorescently labeled and then hybridized to the probes. A successful hybridization event between the labeled target and the immobilized probe results in an increase of fluorescence intensity over a background level, and can be measured using a fluorescent scanner.

Multiplex microarray systems suitable for the purpose of embodiments described herein include, but are not limited to, printed and in situ-synthesized microarrays, high-density bead arrays, electronic microarrays and suspension bead microarrays. A microarray system is non-limitedly exemplified by miRXplore™ Microarray (Miltenyi biotech, Germany) that contains quadruplicate spots of all human, mouse, rat, and viral miRNA sequences.

Universal Reference (UR) is an equimolar pool of about 1000 single-stranded synthetic RNA oligonucleotides matching mature microRNAs that may serve as a reliable and comprehensive microRNA reference in miRNA profiling. Commercially available UR miRNA pools are used as a general reference as well as hybridization control, and quality control for microarray hybridization. A non-limiting example of UR is miRXplore™ Universal Reference (Miltenyi biotech, Germany) that matches the miRXplore™ Microarray system.

A two-color microarray hybridization was used for miRNA profiling in embodiments described herein. In the two-color microarray hybridization described herein, the sample (target) RNA labeled with one fluorescent dye such as cyanine dye5 (Cy5) or its spectrally equivalent Hy5 (both fluoresce in the red region), and universal sequences (corresponding to target miRNAs) labeled with another dye, e.g., cyanine dye3 (Cy3) or its spectrally equivalent Hy3 (both fluoresce greenish yellow), are hybridized on one microarray. The principle of the microarray reference is based on the hybridization of each of several samples versus the reference. Signal ratios are calculated from the ratio of each sample(s) versus universal reference. As the labeled molecules compete for the same probes on the microarray, the hybridization efficiency stays the same.

Microarray analysis was performed on 48 ALL samples using the miRXplore™ microarray platform (Miltenyi biotech, Germany). RNA quality was assessed by Agilent 2100 Bioanalyzer platform (Agilent technologies) and visualized by means of agarose gel electrophoresis. Sample labeling was performed according to the miRXplore™ microarray platform user manual. For those samples which revealed a sufficient RNA yield, 2 pg total RNA were used for the labelling. For all other samples, the available amount of total RNA was used. Test samples were labeled with Hy5, and reference sequences were labeled with Hy3. The miRXplore™ UR was used. Subsequently, the fluorescently labelled RNA sequences were hybridized overnight to miRXplore™ microarrays using the a-Hyb™ hybridization station (Miltenyi biotech, Germany). Fluorescent signals of the hybridized miRXplore™ microarray were detected using a laser scanner of Agilent (Agilent Technologies). Normalized Hy5/Hy3 ratios were calculated for each quadruplicate by PIQOR™ analyzer (Miltenyi Biotech, Germany). Only miRNAs that had a signal that was equal to, or higher than the 50% percentile of the background signal intensities were used for the Hy5/Hy3 ratio calculation. Data was transformed to Log 2 ratios for data clustering (2D-clustering using Pearson correlation and average linkage).

### Quantitative Reverse Transcription PCR (RT-qPCR)

miRNA-microarray results were verified by RT-qPCR on 95 samples. For RT-qPCR studies, 100 ng RNA was converted to complementary DNA (cDNA) using universal cDNA synthesis kit (Exiqon, Vedbaek, Denmark) RT-qPCR was performed using locked-nucleic acid (LNA™) primers sets (Exiqon, Vedbaek, Denmark) and 5S Ribosomal RNA as a reference gene. The RT-qPCR reactions were performed in duplicates on the LightCycler® 480 instrument (Roche, Rotkreuz, Switzerland). The results were expressed as relative expression using the delta-Ct method as described herein.

### Cell Lines and Transient Transfections

The NALM-6 (precursor B-cell ALL) cell line was obtained from the American Type Culture Collection® (ATCC®) and cultured according to ATCC growth recommendation. Two hundred (200) pmol of miR-451 mimic (a dsmiR-451 designed to copy or mimic the functionality of mature endogenous miR-451 upon transfection) (IDT®-Syntezza, Jerusalem, Israel), 200 pmol scrambled miRNA (IDT®-Syntezza, Jerusalem, Israel) serving as miRNA mimic negative control, or 2 μg antagomiR-451 plasmid (GeneCopoeia™, Rockville, Md., USA) serving as miR-451 inhibitor, were transiently transfected by electroporation into NALM-6 cells using the Amaxa®Nucleofector technology (Lonza).

### Cell Proliferation Assay

Cell viability assay was performed using the Cell Proliferation Kit comprising the second-generation tetrazolium dye, XTT. Viable cells with active metabolism convert XTT into an orange colored reduction product, whereas dead cells lose the ability to reduce XTT, thus, color formation serves as a useful and convenient marker of only the viable cells.

The XTT kit was applied to NALM6 transfected cells according to the manufacturer's protocol (Biological Industries, Kibbutz Beit Haemek, Israel). Cells were incubated for 3 hours with the XTT reagent and quantity of reduced XTT (directly proportional to the number of viable cells) was assessed by recording changes in absorbance as measured on a microplate reader at 450-500 nm optical density (OD).

### Luciferase Assay

The luciferase reporter assay is commonly used as a tool to study gene expression at the transcriptional level. Typically, a reporter gene is cloned with a DNA sequence of interest into an expression vector that is then transferred into cells. Following transfection, the cells are assayed for the presence of the reporter by directly measuring the reporter protein itself or the enzymatic activity of the reporter protein. Luciferases make up a class of oxidative enzymes found in several species, for example, Firefly (e.g., Photinus pyralis) and Renilla (Renilla reniformis, a sea pansy) that enable the organisms that express them to “bioluminesce,” or emit light. Firefly luciferase is a very sensitive genetic reporter due to the lack of any endogenous activity in mammalian cells or tissues. Firefly luciferase follows Michaelis-Menten kinetics and, as a result, maximum light output is not achieved until the substrate and co-factors are present in large excess. When assayed under these conditions, light emitted from the reaction is directly proportional to the number of luciferase enzyme molecules. Renilla luciferase is used as a reporter for studying gene regulation and function in vitro and in vivo, and, in accordance with embodiments described herein, it is used as a normalizing transfection control for Firefly luciferase assay. Luciferase reporter assay kits comprising expression vectors that contain the luciferase reporter gene from Firefly or from Renilla (or a variation thereof) and reagents necessary for the reaction to occur, are commercially available, for example the Luc-Pair™ miR Luciferase Assay Kit, a 96-well plate format for measuring Firefly and Renilla luciferases sequentially (manufactures include, for example, GeneCopoeia™).

To perform the reporter assay, the regulatory region of NAMPT is cloned upstream of the Firefly luciferase gene in an expression vector, and the resulting recombinant vector, e.g., pLightSwitch 3′UTR, is transfected into cells along with an expression vector comprising the Renilla gene serving as reference. In accordance with some embodiments described herein, a target reporter vector containing the full-length wild type NAMPT 3′ untranslated region (3′-UTR) (pLightSwithch NAMPT 3′UTR) purchased from GeneCopoeia™ (Rockville, Md., USA) was co-transfected into NALM-6 cells along with Renilla luciferase vector as internal vector control. The NAMPT 3′-UTR contains miRNA response elements (MREs), to one of which miRNA-451 specifically binds. Twenty-four hours before transfection, NALM-6 cells were plated in 6 wells until 70% confluent. Two hundred (200) pmol miR-451 mimic, 200 pmol scrambled miR (both purchased from IDT®-Syntezza, Jerusalem, Israel) or 2 μg antagomiR-451 plasmid (GeneCopoeia™, Rockville, Md., USA) were transiently transfected into NALM-6 cells together with 2 μg pLightSwitch NAMPT 3′UTR and Renilla luciferase vector. Luciferase assay was performed 24 hours after transfection by the Luc-Pair™ miR luciferase assay (GeneCopoeia™, Rockville, Md., USA). Briefly, cells were collected, lysed to release all proteins (including the luciferase), luciferin was added along with all the necessary cofactors, and the enzymatic activity was quantitatively measured using a luminometer. Since NAMPT 3′-UTR was fused to the luciferase reporter gene, the luciferase activity is directly correlated with the activity/expression of NAMPT. For each sample, firefly luciferase activity was normalized to Renilla luciferase activity in the same well.

### Western Blot

Protein was extracted from NALM-6 cell line using Qproteome™ lysis buffer (Qiagen, Hilden, Germany). The membrane was then probed with one or more of the following antibodies: anti-disintegrin and metalloproteinase domain-containing protein 10 (ADAM10) (1:1000) (Abcam; Cambridge, UK) (see Example 9); anti-chemokine C-X-C motif 16 (CXCL16) (1:1000); anti-NAMPT (1:1000); and anti-GAPDH (1:5000) (Santa Crus Biotechnology, Santa Crus, Calif., USA) (see Examples 5 and 9). The secondary antibodies were goat anti-mouse and goat anti-rabbit (1:10,000) (Sigma®, Saint Louis, Mo., USA), for each antigen according to the manufacturer's instructions. Glyceraldehyde-3-phosphate dehydrogenase (GAPDH) was used as a loading control for protein normalization (GAPDH, a so-called housekeeping protein, is one of the key enzymes involved in glycolysis and constitutively expressed in almost all tissues in high amounts).

Intensity of the protein band was measured using an in-house program. Then, each value was divided by the value obtained for GAPDH from the same sample. The normalized ratio expressed the protein expression level in the sample. The protein level of a control sample was adjusted to 100% and test protein expression level is indicated as % relative to control.

### NAMPT Protein Expression Measurement by Flow Cytometry (Fluorescence-Activated Cell Sorter (FACS))

One to two million NALM-6 transfected cells were collected 48 hours after transfection and fixed using 500 μl fixation and permeabilization buffer prior to immunofluorescent staining of intracellular proteins (e.g., BD Phosflow™ Fix Buffer I (BD Biosciences, San Jose, Calif., USA)) for 15 minutes. Then, pellets were washed twice using PBS and resuspended in 500 μl methanol. Samples were incubated for 2 hours in −20° C. For staining, pellets were washed with PBS and resuspended in 10 ml BD™ Cell Wash buffer (BD Biosciences, San Jose, Calif., USA) containing 2% FCS. The cells were stained for NAMPT using anti-NAMPT antibody (Ab) (1:1000) (R&D Systems®, MN, USA). The secondary antibody was goat anti-sheep (1:10,000) (R&D Systems®, MN, USA). The cells were sorted on flow cytometer (Calibur™ flow cytometer, Becton Dickinson, Le Pont-De-Claix, France) and analyzed using BD CellQuest™ Pro software.

NAD⁺/NADH Ratio Assay (NAD⁺ Assay)

The NAD⁺/NADH ratio was measured in whole-cell extracts of NALM-6 cells using the Biovision NAD⁺/NADH Quantification Colorimetric™ Kit (BioVision, San Francisco, Calif., USA) (also referred to herein as “NAD assay”) according to the manufacturer's protocol (see, for example, https://www.biovision.com/documentation/datasheets/K337.pdf). The NAD Cycling Enzyme Mix in the kit specifically recognizes NADH/NAD⁺ in an enzyme cycling reaction. After 4 hrs, the plate was read at optical density (OD) 450 nm. The readings were normalized to the cell number and the NAD⁺/NADH ratio was calculated as: (total NAD⁺−NADH)/NADH.

### Apoptosis Assay

Cells were stained with annexin V-fluorescein-5-isothiocyanate (FITC) conjugated antibody and propidium iodide (PI) using the Annexin V-FITC Apoptosis Detection Kit (ab14085, Abcam® Incorporated, Cambridge, Mass., USA) according to manufacturer's instructions (see, for example, https://www.abcam.com/ps/products/14/ab14085/documents/ab14085%20Annexin %20V%20FITC%20Apoptosis%20Detection%20Kit%20v4%20(website).pdf), and read on the flow cytometer. Annexin V Apoptosis Detection Kit is based on the observation that soon after initiating apoptosis, cells translocate the membrane phosphatidylserine (PS) from the inner face of the plasma membrane to the cell surface. Once on the cell surface, PS can be easily detected by staining with a fluorescent conjugate (e.g., FITC) of Annexin V, a protein that has a high affinity for PS. The one-step staining procedure takes only 10 minutes. When performing both Annexin V-FITC and PI staining, it is possible to differentiate apoptosis vs necrosis.

### Stable Lines of Transduced NALM-6 Cells

miR-451 precursor and antagomiR-451 expression clones were obtained from GeneCopoeia™ (GeneCopoeia, Rockville, Md., USA). Stable cell lines were prepared by the Tel Aviv University's Vector Core Facility by transduction of ALL cell line (NALM-6) with lentiviral particles co-encoding miR-451 precursor or antagomiR-451, the ORF for EGFP/mCherry and puromycin-resistance, under the control of individual CMV promoters. The ORFs and the miR-451 precursor/antagomiR-451 oligonucleotide sequences were incorporated into the pLL3.7 3rd generation lentiviral vector. Lentiviral particles were produced from these vectors by calcium-phosphate mediated co-transfection thereof to HEK 293FT cells together with the helper plasmids pMDLg-RRE, pRSV-Rev and VSV-G. Forty-eight hours after transfection, the medium was collected, filtered and transferred to flasks containing low density NALM-6 cells in suspension. Three days after viral transduction, puromycin (3 μg/ml) was added to the media in order to sort out non-transduced cells.

### In-Vivo Xenograft Mice Model

Ten million ALL NALM-6 cells in 100 μl saline were injected subcutaneously, above the femur, to 6-8 weeks-old NSG™ mice (NSG™ mouse model variants are the most highly immunodeficient mice and therefore were chosen as models for cancer xenograft modelling). Tumors were clinically evident 15-28 days following injection in more than 85% of the mice (n=175). Mice were divided into groups receiving either FK866 or vehicle treatment. Mice in the treatment group received FK866 in 0.9% saline intraperitoneally at a dose of 15 mg/Kg daily for 5 days. Vehicle in the control group was injected intraperitoneally once a day for 5 days repeated weekly. The volume of tumors was measured daily until mice were sacrificed. Tumor volume was monitored using vernier caliper measurements and calculated using the formula π(length)(width)²/6. Each experiment was ended when tumor size reached 12×12 mm³. All animal experiments were performed under an approved animal study protocol.

### Statistical Analyses

miR expression data were analyzed with PASW Statistics 18 or 21 (SPSS inc. Chicago, Ill., USA). For correlation with age, gender, WBC, prednisone response (d8), type and/or risk group, the Fisher's exact test was used. In order to determine the optimal cut-off value, receiver operating characteristic (ROC) curve analysis was performed for each miRNA. Kaplan-Meier analyses were performed to evaluate whether the selected miRNA correlates with relapse, and COX regression analysis was used to determine whether tested miRNAs can be regarded as independent risk factors. A p-value (P)<0.05 was considered as significant for the survival analyses. For miRNA-451 studies described in Example 6 herein, the variants used for Cox proportional hazards regression models were expression of the miR-451, age, WBC, prednisone response, and IKZF1, ETV6-RUNXJ translocation status.

A repeated-measures analysis of variance (ANOVA) was performed to investigate the drug effect over time in different miR-451 expression groups (Examples 9 and 10 herein). Data were analyzed using BMDP Statistical Software (1993, Chief Editor: W J Dixon, University of California Press, Los Angeles, Calif., USA). Other statistical analyses were performed using Student's t-test. A 2-sided p-value <0.05 was considered significant.

### Example 1

### miR-151-5p, miR-451 and miR-1290 Expressions Correlate with Various ALL Clinical Parameters

This example shows that ALL prognosis can be accurately predicted by decreased expression of the miR-151-5p and miR-451, accompanied by an increased expression of miR-1290.

Microarray analysis was used to determine ALL-specific miR expression profile as described in Material and Methods above. From a panel of 979 synthetic miRNA, only 116 sample miRNAs were significantly higher, and 116 sample miRNAs were significantly lower, relative to the universal reference (UR) sequences. Clustering with age, type, WBC, d8, risk group or relapse revealed 10, 33, 20, 14, 19 and 33, respectively, miRNAs that were significantly lower expressed in ALL, while 9, 36, 16, 12, 14 and 28 (respectively) miRNAs were significantly higher expressed in ALL. Analysis of the lower-expressed miRNAs was described in International Publication No. WO 2012/042516, the entirety of which is incorporated by reference herein. Therein, it was described that combined decrease in miR-151-5p and miR-451 expression is predictive of increased risk of ALL relapse and worse disease prognosis.

In a further analysis of the miR-ALL microarray, 4 miRNAs were chosen that were upregulated and associated with at least 3 adverse prognostic markers: miR-196b, miR-424, miR-1248, and miR-1290. To confirm the correlation between expression and ALL, the expression levels of these 4 miRNAs were further analysed by quantitative reverse transcription PCR (RT-qPCR) in a cohort of 125 pediatric ALL patients (B-cell ALL and T-cell ALL). Of the 4 miRNAs analysed, only miR-1290 significantly correlated with ALL outcome.

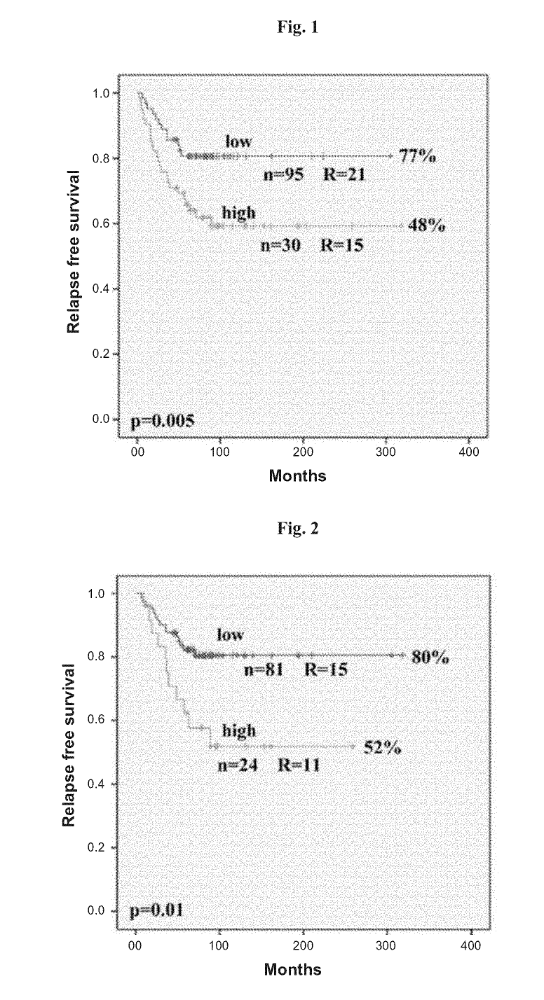

Using quartile 3 (Q₃; the median of the upper half of the data set. About 75% of the numbers in the data set lie below Q₃ and about 25% lie above Q₃) as a cut-off, patients expressing high levels of miR-1290 had a 48% relapse free survival (RFS) versus 77% RFS in those expressing low levels of the miRNA (p=0.005; FIG. 1). When the median expression level was used as the cut-off, the significant correlation with outcome was maintained. RFS was 59% for those expressing high levels versus 81% for those expressing low levels of the miRNA (p=0.017, data not shown). A significant correlation with outcome was also observed when analyzing only the B-lineage ALL patients (n=105). Patients expressing high levels of miR-1290 had a 52% RFS versus 80% for those expressing low levels (p=0.010; FIG. 2).

When applying multivariate Cox regression analysis with the variants: miR-1290 expression level, age, WBC, and prednisone response (d8) in the B-lineage cohort, both miR-1290 expression level and WBC were identified as significant independent prognostic markers. From this analysis, it was determined that a patient expressing high levels of miR-1290 has a 3-fold increased risk of relapse (Table A).

TABLE A
Multivariate Cox regression analysis for relapse in the
B-lineage cohort (n = 105)

| | Univariate | Multivariate | | |
|---|---|---|---|---|
| Variant | P | P | HR | 95% CI |
| miR-1290 high vs | 0.010 | 0.006 | 3.03 | 1.4-6.6 |
| low expression | | | | |
| Age 1 to 6 vs | NS | | | |
| <1 or >6 years | | | | |
| WBC below vs | 0.001 | 0.001 | 3.8 | 1.7-8.4 |
| above 20 × 10⁹ cell/L | | | | |
| Prednisone response | NS | | | |
| poor vs good | | | | |

HR—hazard ratio;
CI—confidence interval, the probability that a value will fall between an upper and lower bound of a probability distribution. Namely, the range of values which is likely to contain the true HR. Confidence intervals are constructed at a confidence level of, for example, 95%, namely, if the same population is sampled on numerous occasions and interval estimates are made on each occasion, the resulting intervals would bracket the true HR parameter in approximately 95% of the cases;
P—p-value;
NS—not significant.

Currently, the risk of ALL relapse is based on the detection of minimal residual disease (MRD) following treatment on days 33 and 78 from diagnosis using the PCR-MRD assay described herein. The amount of residual leukemic cells determines the risk groups and treatment is adjusted accordingly. The aim is to increase treatment in the high-risk group and reduce in the favourable group. Multivariate Cox regression analysis was applied again including the MRD data, which was available for 61 B-lineage ALL patients. All patients excluding 2, were MRD non-high-risk patients. A patient expressing high levels of miR-1290 had an increased risk fold of 4.8 to relapse (p=0.027; Table B).

TABLE B
Multivariate cox regression analysis for relapse in the B-lineage
minimal residual disease (MRD) non-high-risk cohort (n = 6l)

| | Univariate | Multivariate | | |
|---|---|---|---|---|
| Variant | P | P | HR | 95% CI |
| miR-1290 high vs | 0.014 | 0.027 | 4.8 | 1.2-19.5 |
| low expression | | | | |
| Age 1 to 6 vs | NS | | | |
| <1 or >6 years | | | | |
| WBC below vs | 0.037 | 0.055 | 4.1 | 0.1-17.2 |
| above 20 × 10⁹ cell/L | | | | |
| Prednisone response | NS | | | |
| poor vs good | | | | |
| MRD | NS | | | |

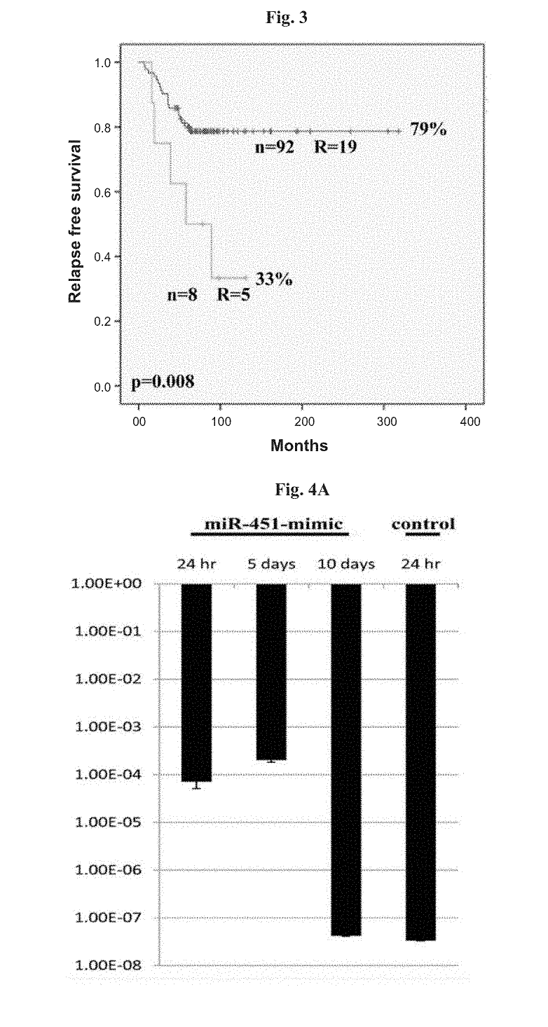

When data related to the downregulated and upregulated miRs was combined, it was revealed that the patients expressing low levels of both miRNAs (miR-151 and miR-451) with high levels of miR-1290 had a very poor outcome: 33% versus 79% RFS for all other combinations (p=0.008; FIG. 3).

When Multivariate Cox regression analysis was applied to the risk of relapse in the combined results of the down and up-regulated miRNAs for a cohort of non-high risk it was shown that a patient expressing low levels of both miR-151 and miR-451 with high levels of miR-1290 was, in fact a high-risk patient that had an increased risk of 16.7 to relapse (p=0.006; Table C).

TABLE C
Multivariate Cox regression analysis for relapse in the
PCR-MRD non-high risk cohort (n = 54)

| | Univariate | Multivariate | | |
|---|---|---|---|---|
| Variant | P | P | HR | 95% CI |
| Combination of all 3 | 0.02 | 0.006 | 16.7 | 2.3-122 |
| miRNAs | | | | |
| Upregulated miR-1290 | 0.021 | | | |
| Both miR-151 and | 0.06 | | | |
| miR- | | | | |
| 451 downregulated | | | | |
| Age 1 to 6 vs | NS | | | |
| <1 or >6 years | | | | |
| WBC below vs | 0.037 | 0.055 | 4.1 | 0.1-17.2 |
| above 20 × 10⁹ cell/L | | | | |
| Prednisone response | NS | | | |
| poor vs good | | | | |
| MRD | 0.075 | 0.017 | 6.9 | 1.4-33 |

Based on these analyses it can be concluded that by combining detection of miR-151, miR-451, and miR-1290 together, very high-risk patients can be accurately detected within a cohort of non-high-risk patients, so that those patients at high risk of relapse could benefit from a more intensive therapy, already at the time of diagnosis.

### Example 2

### Up-Regulation of miR-451 Decreases ALL Cells Growth

As described herein, decreased expression of miR-451 in comparison to a control level can serve as a prognostic factor for ALL relapse risk, as low expression of miR-451 at first diagnosis predicts worse outcome. To demonstrate the effect of miR-451 in ALL, miR-451 was up regulated in ALL derived Nalm-6 cell line using miRNA-451 mimic (SEQ ID NO:2) transfection (Nalm-6/miR-451 mimic) by electroporation (Amaxa® Nucleufector® Technology; kit T; program c-005). Scrambled miRNA-transfected cells served as negative control (Nalm-6/miR-NC). Quantitative reverse transcription PCR (RT-qPCR) was used to confirm miR-451 expression in the transfected cells.

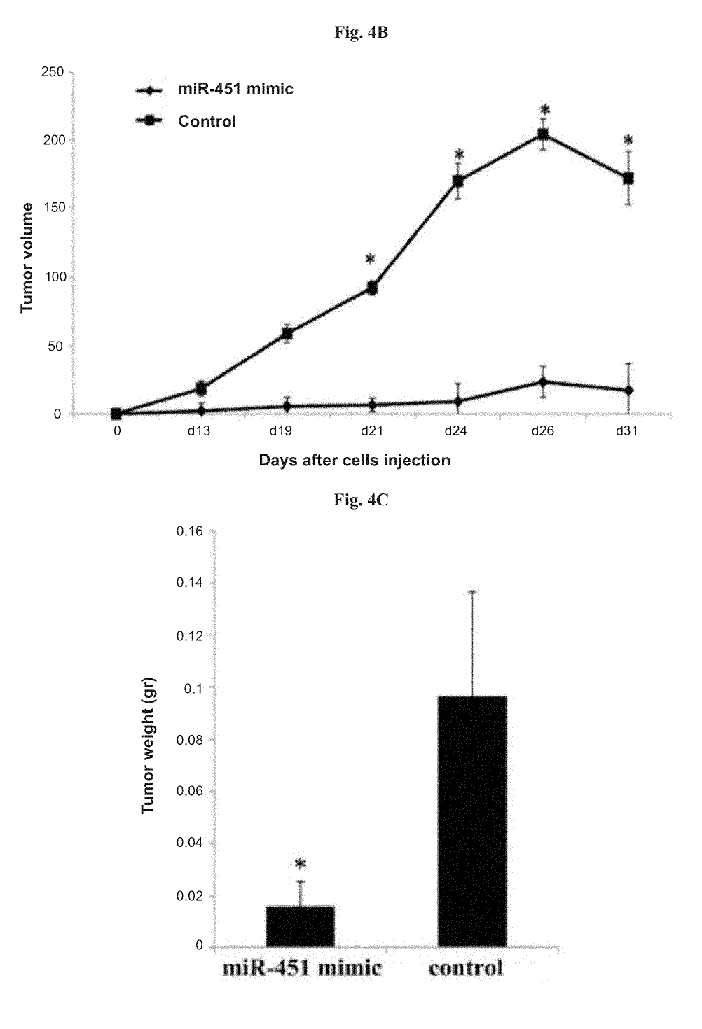

The RT-qPCR results, summarized in FIG. 4A, show a significant increase in the expression of hsa-miR-451 in Nalm-6/miR-451 mimic versus the negative control cells (Nalm-6/miR-NC), 24 hours and 5 days after transfection (hsa-miR-451 is the miR-451 produced by the miR-451 mimic transfected cells).

To further study the putative tumor-suppressive function of hsa-miR-451 in vivo, 10⁷ viable Nalm-6 cells either untransfected, transfected in vitro with miR-451 mimic, or transfected with scrambled miR (control), were injected sub-cutaneously (s.c.) into the right flanks of 6-week-old female NOD/SCID mice. Whereas animals transplanted with scrambled miR control cells developed large tumors after 20 days, animals receiving Nalm-6/miR-451 mimic cells showed significantly decreased tumor growth (FIG. 4B). On day 26, the median tumor volume in the scrambled control mice and the miR-451 mimic mice were 204.69 mm³ (SE=63.96) and 23.32 mm³ (SE=13.12), respectively (p=0.019). At the end of the experiment, mice were sacrificed, and the tumors were weighted. The median tumor weight in the scrambled control mice and the miR-451 mimic mice were 0.0966 gr (SE=0.040) and 0.0159 gr (SE=0.0009), respectively (p=0.046) (FIG. 4C).

These results indicate that up regulation of miR-451 mediates cell growth in ALL and supports the role of miR-451 as a tumor suppressor gene.

### Example 3

### miR-451 Inhibits NAMPT Expression in ALL Cell Lines by Targeting NAMPT 3′-UTR

This example demonstrates that the NAMPT mRNA is a specific target of miR-451 translation inhibition.

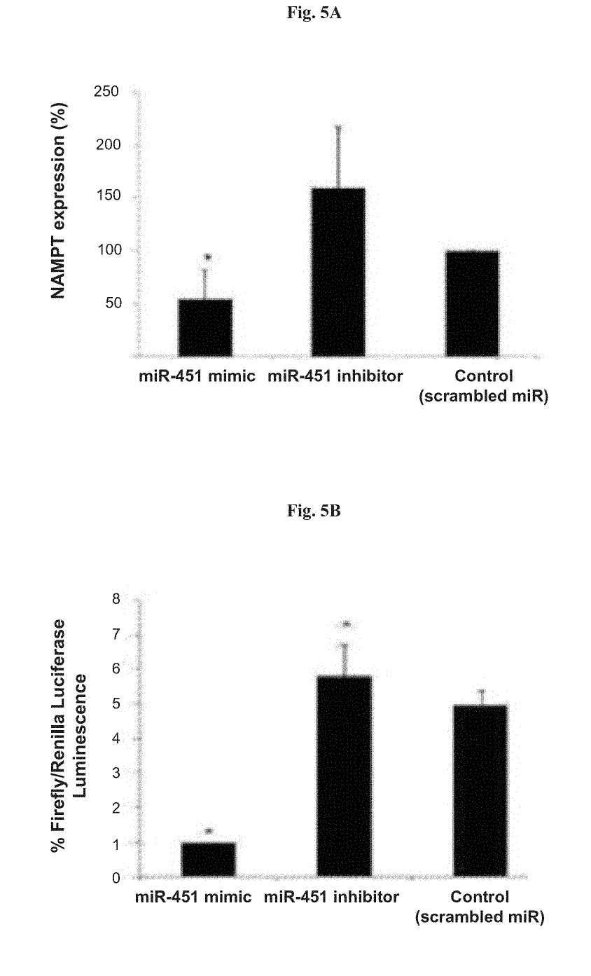

Using open access software programs (TargetScan and miRanda), NAMPT was identified as a predicted target of miR-451. To determine the effects of miR-451 on NAMPT expression, Nalm-6 cells were transfected with either miR-451 mimic (SEQ ID NO: 2), miR-451 inhibitor (miArrest™ miR-451, an inhibitor expression clone; GeneCopoeia™) or scrambled miR (commercial universal sequence; serving as negative control). NAMPT expression was measured by FACS analysis using a specific NAMPT antibody. Following the over-expression of miR-451 in Nal-6/miR-451 mimic cells, NAMPT protein expression was decreased by 46% while miR-451 inhibitor caused a 60% increase in NAMPT expression in Nalm-6/miR-451 inhibitor cells (FIG. 5A, p<0.05).

To confirm that NAMPT is a direct target of miR-451, luciferase reporter vectors were purchased that contained the NAMPT 3′-UTR (LightSwitch™ NAMPT 3′UTR Reporter; GoClone®). Luciferase reporter assays (LightSwitch™ Luciferase Assay; SwitchGear Genomics) were then performed in the presence and absence of miR-451 mimic and miRNA-451 inhibitor to determine whether NAMPT was a direct downstream target of miR-451. The relative luciferase activity of the reporter that contained NAMPT 3′-UTR was decreased by 80% following miR-451 mimics transfection. In contrast, miR-451 inhibitor transfection showed a significant 17% increase in the relative luciferase activity of the reporter (FIG. 5B, p<0.05). These results confirm that miR-451 directly binds the 3′-UTR of NAMPT transcript, and negatively regulates its protein levels.

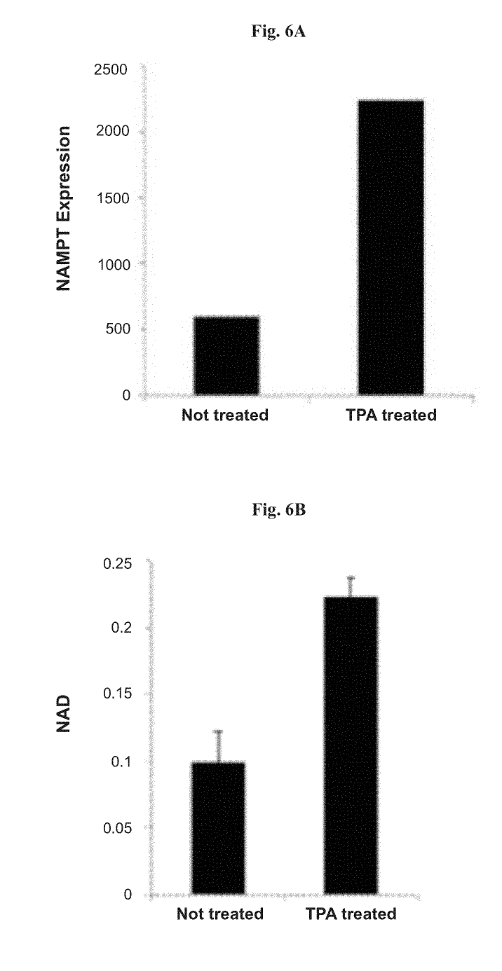

Studies by several investigators have shown that 12-0-tetradecanoylphorbol-13-acetate (TPA) (Sigma) is an extraordinarily potent tumor promoter and stimulates protein kinase C (PKC). Since NAMPT is over-expressed in several tumors, it was believed that it might be possible to achieve NAMPT stimulation by TPA treatment. To test this hypothesis, human peripheral blood cells were treated with 50 ng/ml TPA for 24 hours, and NAMPT expression was measured by FACS using a specific NAMPT antibody. Cells treated with TPA showed an increase of more than 4 folds in NAMPT expression levels (FIG. 6A).

NAMPT is the rate-limiting enzyme in the NAD⁺ biosynthetic pathway. Thus, NAD⁺ levels in the stimulated cells were measured using a standard NAD assay (BioVision NADH/NAD⁺ Quantification Colorimetric™ Kit). It was found that the cellular NAD⁺ levels in the TPA stimulated cells were 2-fold higher (FIG. 6B).

### Example 4

### Increased Expression of NAMPT Increases Sensitivity of ALL Cells to the NAMPT Inhibitor FK866

Example 3 herein shows that miR-451 regulates NAMPT expression, and by extension, cellular NAD⁺ levels. This example demonstrates that ALL cells in which miR-451 expression is decreased have increased sensitivity to the NAMPT inhibitor FK866.

FK866 is a potent NAMPT inhibitor that is known to cause the depletion of intracellular NAD⁺ levels in the cells and ultimately induce apoptosis. The effect of FK866 treatment of Nalm-6 cell line on apoptosis and NAD⁺ levels was thus characterized.

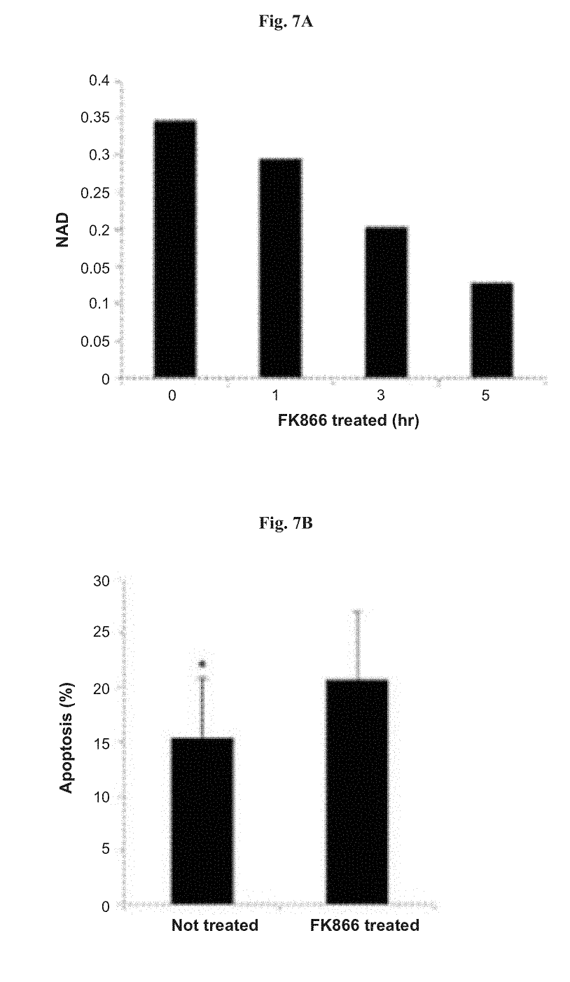

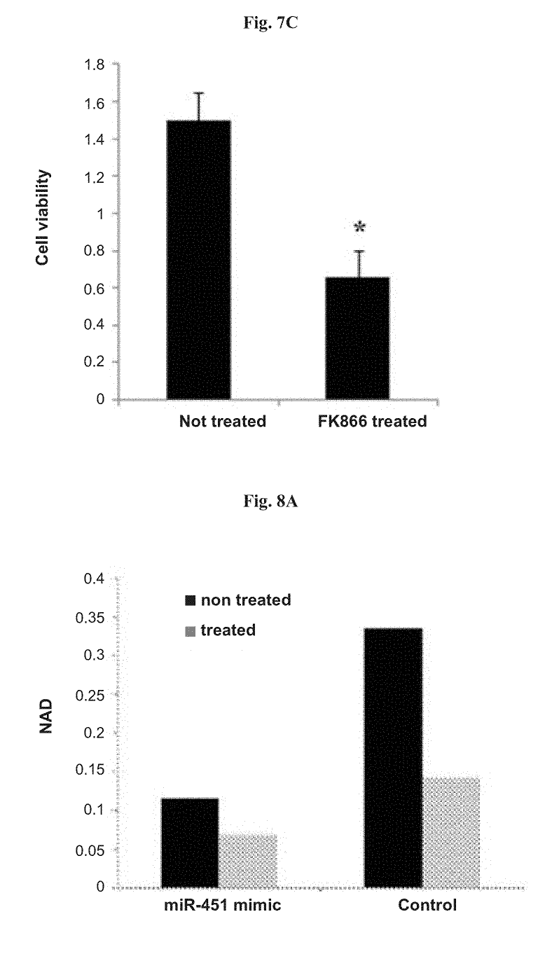

Nalm-6 cells were treated for 1, 3, and 6 hours with 1 nM FK866 (Sigma) and NAD⁺ formation was measured using NAD assay as described in Materials and Methods. The results show a gradual decrease in NAD⁺ detection following FK866 treatment (FIG. 7A). Hence, FK866 is a specific inhibitor of NAD⁺ formation in Nalm-6 cell line.

To measure the effect of NAD⁺ depletion following FK866 treatment of Nalm-6 cells, apoptosis and viability were measured as described in Materials and Methods. Nalm-6 cells were treated for 48 hours with 1 nM FK866, and then apoptosis was measured using FACS. As shown in FIG. 7B-7C, cells treated with the NAMPT inhibitor showed a significantly increased apoptosis level (t-test; p=0.013) (FIG. 7B), and a significant decrease in cell viability (t-test; p=0.0045) (FIG. 7C).

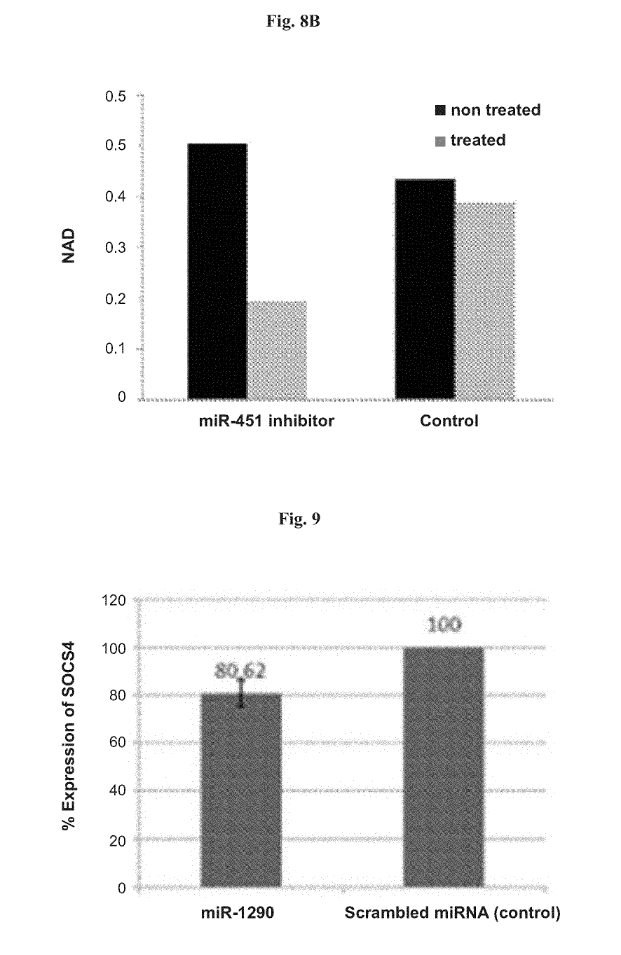

The sensitivity of NALM-6 cells to the NAMPT inhibitor FK866 was measured in cells following transfection with miR-451 mimic (SEQ ID NO: 2), miR-451 inhibitor (miArrest™ miR-451 expression clone; GeneCopoeia™), or scrambled miR. Sensitivity was assessed by measuring the levels of NAD⁺. Nalm-6/miR-451 mimic cells showed less change in NAD⁺ production after FK866 treatment compared to control (scramble miR cells) (FIG. 8A). However, Nalm-6/miR-451 inhibitor cells showed more than 5-fold change in NAD⁺ production after FK866 treatment compared to control (FIG. 8B; p=0.003). These results suggest that ALL cells expressing low levels of miR-451 are more sensitive to NAMPT inhibitors. Thus, miR-451 expression can distinguish between patients that could benefit from treatment with NAMPT inhibitors such as FK866.

### Example 5

### miR-1290 Targets Expression of SOCS4

This example describes the determination of SOCS4 as a target of miR-1290, which will be affected by the miR-1290 overexpression observed in ALL subjects with a higher rate of relapse.

Regulation of the hematopoietic system and the immune response is largely mediated by cytokines. Cytokine signalling is initiated through ligand interaction with specific trans-membrane receptor subunits. The subsequent receptor oligomerisation results in activation of either an intrinsic kinase domain or receptor associated Janus kinases (JAKs), and the following cascade of intracellular phosphorylation and signal transduction culminates in an appropriate cellular response. This cascade is exquisitely cellular controlled by a multiple-tier control, as loss of regulation can promote tumorigenesis and chronic inflammation. Signal transducer and activator of transcription (STAT) proteins are a family of cytoplasmic transcription factors consisting of 7 members that are activated by receptor associated JAKs. Proteins of the STAT-induced STAT inhibitor (SSI), also known as suppressor of cytokine signalling (SOCS) family, are cytokine-inducible negative regulators of cytokine signalling and negative feedback regulators of the JAK-STAT signal transduction pathway. About eight members of the SOCS family gene have been identified. The Socs4 gene encodes the SOCS4, which, like other members in this family, negatively regulates the STAT family. The expression of this gene is induced by various cytokines, including IL6, IL10, and interferon (IFN)-gamma. The protein encoded by this gene can bind to JAKs such as JAK2 and inhibit their activity. The JAK2 kinase in known to be activated in leukemia.

Using target prediction softwares (e.g., miRDB, miRANDA), SOCS4 was chosen as a potential target of miR-1290.

Following transfection of Nalm6 cell line with either miR-1290 mimic (SEQ ID NO:4, over-expression), miR-1290 inhibitor (antisense SEQ ID NO:5, silencing), or scrambled miR (control), the protein levels of SOCS4 were measured using Western blotting (p=0.029; FIG. 9). The values shown in the figure are mean±S.D from 3 experiments.

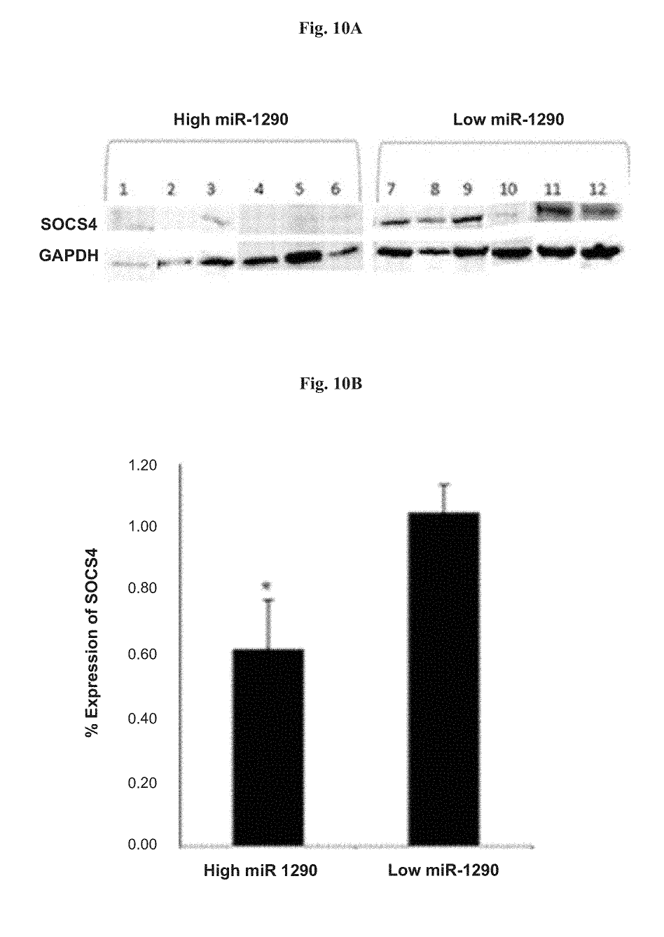

Additionally, SOCS4 protein levels were measured in 31 BM samples of ALL patients and compared to the levels of miR-1290. SOCS4 protein levels were significantly reduced in the samples harbouring high miR-1290 expression levels (p<0.0001; FIGS. 10A-10B).

Phosphorylated STAT (phospho-STAT, activated by JAK) levels were measured by FACS analysis following transfection of miR-1290 mimic (overexpression) into NALM-6 cell line. An increase of 50% in the levels of phospho-STAT protein was evident in the cells expressing high levels of miR-1290 (FIG. 11).

JAK2 is an essential gene in the leukemic process. It has been shown herein that the overexpression of miR-1290 results in the down-regulation of SOCS4. SOCS4 normally inhibits the activity of JAK2, thus its down-regulation results in the increased activity of JAK2, with no need for external signals (as cytokines).

This result suggests that the expression levels of miR-1290 may predict the presence or absence of an activated JAK/STAT pathway and predict who may benefit from JAK2 inhibitors.

### Example 6

### miR-451 Alone has a Predictive Value for Relapse in ALL Patients

In accordance with Example 1 above, miR-451, as part of a three-miRNAs panel, can predict relapse in children with ALL. For evaluating a putative role for miR-451 as a sole biomarker for early identification of ALL patients who may benefit from NAMPT inhibition, 138 pediatric precursor B-cell ALL patients (age range 0.3-19 years) were retrospectively studied using RNA samples extracted from bone marrow (BM) aspirates (liquid sample of bone marrow tissue removed by suction) at the time of first diagnosis, containing at least 80% leukemic blasts (109 patients were from Schneider Children's Medical Center of Israel (SCMCI), and 29 patients were from Charles University Prague, Czech Republic). The patients were treated based on ALL-Berlin-Frankfurt-Munster (ALL-BFM) protocols. The studies were approved by the local and national ethical committees and all patient material was obtained according to the Declaration of Helsinki. Samples from Philadelphia chromosome-positive (Ph+) ALL patients and known minimal residual disease high risk patient identified or detected by PCR-MRD were excluded from the cohort. The B-cell ALL cohort that included 61 patients 1 year old and younger (infants), and older than 6 years of age (children and adolescents). Forty-five patients (33%) had a white blood cells count (WBC) >20×10⁹/L, and 8 patients (6%) were poor prednisone responders. Thirty-three (24%) patients relapsed. Thirty-nine (28%) patients harboured the ETV6-RUNX1 translocation and two patients the t(4;11)(q21;q23) translocation (MLL-AF4). Five patients were hypodiploid and 46 were hyperdiploid. The median follow-up was 90 months (ranging from 8 to 361 months). Risk groups in the BFM-based protocols relied mostly on an early response to treatment detected in peripheral blood at day 8 of prednisone monotherapy, and with the presence of Ph+ chromosome or t(4;11)(q21;q23) translocation. Eighteen (13%) patients were clinically classified as BFM high risk (HR), 70 (51%) as medium-risk (MR) and 50 (36%) as standard-risk (SR).

To determine a role for miR-451 expression level as a sole indicator of relapse, the ALL samples were analyzed for miRNA expression level by RT-qPCR as described in Materials and Methods.

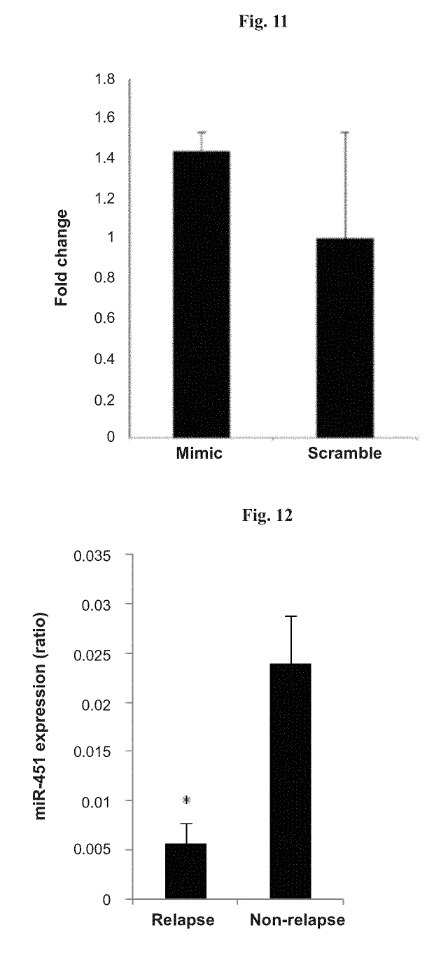

The results presented in FIG. 12 show that miR-451 was significantly decreased in patients that relapsed versus patients in long-term remission (range 57-361 months). Mean expression level of miR-451 in the relapse group was 0.0056±0.0020 whereas mean expression level of miR-451 in the non-relapse group was 0.024±0.004 (p=0.000692).

To further evaluate the predictive value of miR-451, a Cox proportional hazard regression model for relapse was used, taking into consideration the variables available within one week of diagnosis, namely, age, WBC, prednisone response, IKZF1, ETV6-RUNX1 translocations status and miR-451 expression. The reciprocal translocation t(12;21)(p13;q22) is the most frequent chromosomal rearrangement in childhood B-cell precursor ALL with an incidence of ˜25%. The resulting TEL-AML1 (syn.: ETV6-RUNX1) gene expression leads to expansion of B-cell precursors with enhanced self-renewal capacity and impaired differentiation to more mature B-cell stages. IKZF1 deletions and mutations identify high risk biological subsets of ALL.

Values of variables above the first quartile (Q1; the middle number between the smallest number and the median of the data set) were considered as ‘high’ while values below the first quartile were considered as ‘low’. The three variables: IKZF1, ETV6-RUNX1 status and miR-451 expression were identified as independent prognostic markers with a hazard ratio for relapse of 10.847, 0.044 and 11.264, respectively (p=0.002, 0.008 and 0.001, respectively) (Table D).

TABLE D
Univariate and multivariate Cox regression analyses for
ALL relapse

| | Univariate | Multivariate | | |
|---|---|---|---|---|
| Variant | P | P | HR | 95% CI |
| WBC below vs. | NS | | NS | |
| above 20 × 109/L | | | | |
| Prednisone response | NS | | NS | |
| good vs. poor | | | | |
| miR-451 | 0.016 | 0.001 | 11.264 | 2.7-46.7 |
| high vs. low | | | | |
| Ikaros | 0.008 | 0.002 | 10.847 | 2.46-47.9 |
| Wild type vs. IKZF1 | | | | |
| deletion | | | | |
| TEL-AML1 | 0.036 | 0.008 | 0.044 | 0.004-0.44 |
| Translocation vs. | | | | |
| normal | | | | |

HR—hazard ratio;
CI—confidence interval;
NS—not significant;
P—p-value.

Based on the Cox regression analyses it can be concluded that evaluation of miR-451 levels alone, at the day of first diagnosis, affords accurate prediction of high-risk patients within a cohort identified by other means (WBC, d8) as medium- to low-risk patients, thereby enabling to tailor a treatment modality that will effectively prevent relapse in those patients who are truly at high risk of relapse.

### Example 7

### miR-451 has a Role in ALL Cell Growth

The effect of miR-451 expression level on ALL tumor cells was evaluated in in-vitro and in-vivo systems. First, the effect was analyzed in-vitro in NALM-6 cells transiently expressing miR-451 mimic or antagomiR-451 (GeneCopoeia™). No effect on viability and apoptosis rate was observed (data not shown). Then, the effect of miR-451 expression levels on tumor cell growth was evaluated in an in-vivo xenograft mice model. This in vivo study was done in further support of the results described in Example 2 herein. NALM-6 miR-451 mimic and antagomiR-451 stable NALM-6 lines (NALM-6/miR-451 mimic and NALM-6/antagomiR-451, respectively) were prepared as described herein and validated by RT-qPCR for miR-451 expression levels, revealing a level in NALM-6/miR-451 mimic twice as high as that of NALM-6/antagomiR-451 (data not shown). Ten million cells harboring antagomiR-451 or miR-451 mimic were injected s.c. into the right flanks of 6-8 weeks-old NSG™ mice in three independent experiments on a group of 43 mice. Twenty-two mice were injected with the antagomiR-451 stable line, and 21 with the miR-451 mimic line. The volume of the tumors was measured daily from the day tumors were clinically evident in all mice, namely, within 15-25 days from s.c. injection (referred to herein as “day 0”). Tumor growth was calculated as relative tumor growth compared to tumor size at day 0.

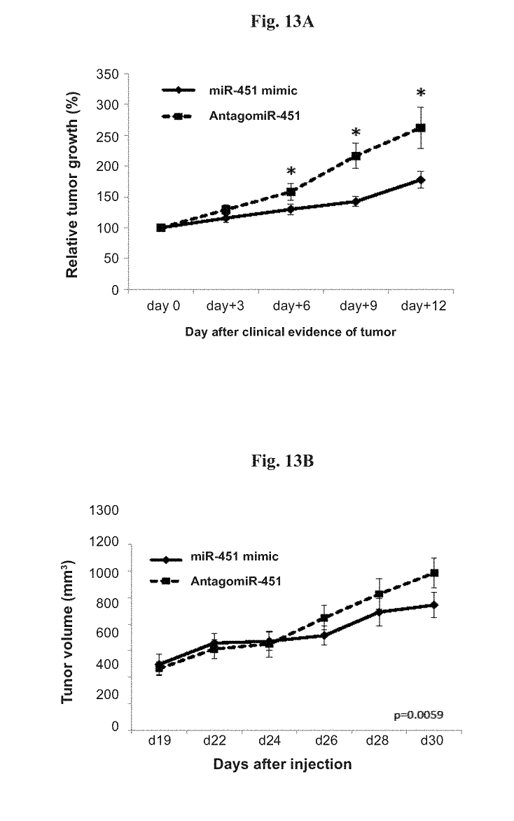

The growth curve summarizing data from all three experiments and representative tumor volume measurements are presented in FIGS. 13A and 13B, respectively. As seen in FIG. 13A, six days after all tumors were first clinically evident, a significant increase in relative tumor growth was obtained in the NALM-6/antagomiR-451 injected mice compared with NALM-6/miR-451 mimic. As seen in FIG. 13B, a significant increase in tumor volume over time was observed in the NALM-6/antagomiR-451 injected mice. Thus, ALL inflicted mice treated with miR-451 inhibitor demonstrated an elevated tumor growth rate compared with mice overexpressing miR-451.

### Example 8

### NAMPT is a Target of miR-451

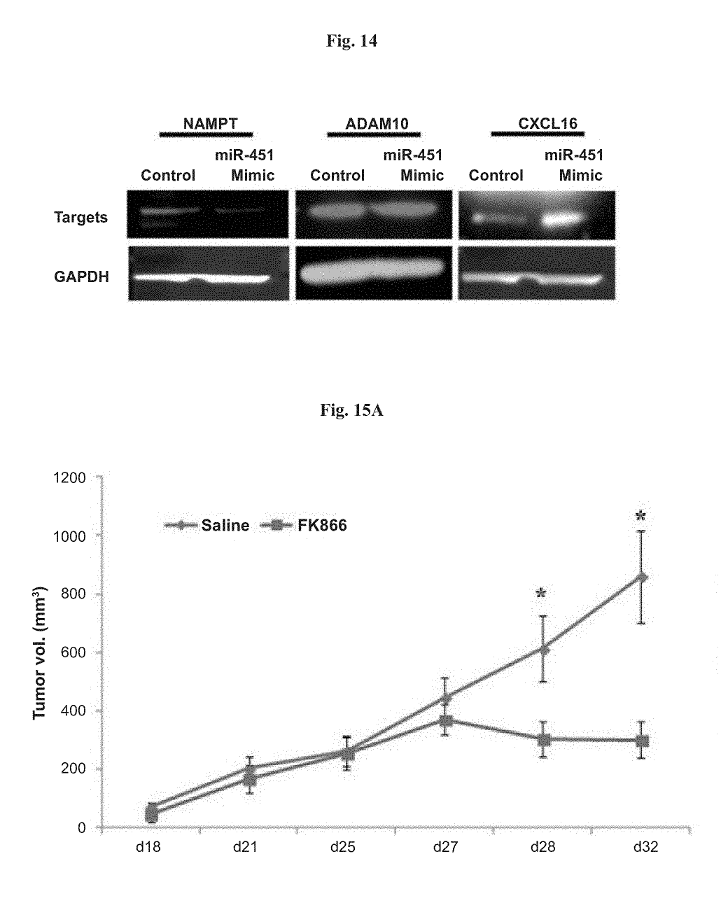

Possible miR-451 targets were evaluated using several open access software programs (e.g., Targetscan, PicTar, miRanda, miRDB). Many optional targets for miRNA-451 action were postulated, of which 3 were chosen that are often upregulated in cancer and associated with tumor growth: anti-disintegrin and metalloproteinase domain-containing protein 10 (ADAM10), a cell surface protein with a unique structure possessing both potential adhesion and protease domains, functioning primarily to cleave membrane proteins at the cellular surface; chemokine C-X-C motif 16 (CXCL16), a small cytokine belonging to the CXC chemokine family; and NAMPT. In the first screening, the suppression of ADAM10, CXCL16 and NAMPT protein level was analyzed in NALM-6 cells transiently expressing miR-451 mimic (SEQ ID NO: 2) and in cells transiently expressing scrambled-miR (universal sequence; negative control) by Western blot as described in Materials and Methods. As seen in FIG. 14, only NAMPT protein expression levels were reduced in the miR-451 mimic compared to control cells (scrambled-miR). About 87-95% of NALM-6 cells expressed NAMPT protein. This result was also validated by FACS analysis (data not shown), and supports the results described in Example 3 herein.

### Example 9

### ALL Tumor Growth is Sensitive to a NAMPT Inhibitor

Following the in vitro studies described in Example 4 herein, the sensitivity of ALL tumor growth to a NAMPT inhibitor was tested in-vivo in an ALL xenograft mouse model. The NAMPT inhibitor tested was the known small molecule FK866. The effect of FK866 was tested in the ALL xenograft mouse model in 4 independent experiments each performed on a group of 82 NSG™ mice as follows: 41 mice were treated with FK866 (15 mg/kg) once a day for 5 days repeated weekly, and 41 mice served as controls (i.e., were not treated with FK866 and received saline instead).

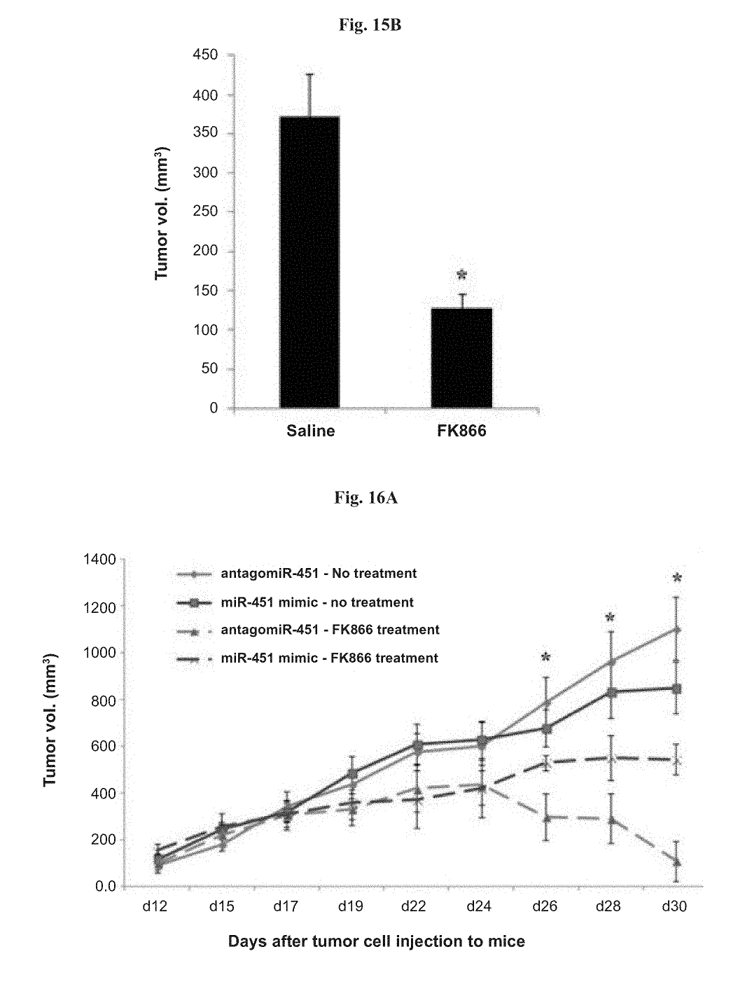

Results of a representative in vivo study are shown in FIG. 15A. Mice were treated from the day tumors were clinically evident in all mice (day 25), and tumors volume was measured daily. Within 4 days of treatment (day 29), a significant difference in volume change was observed between the treated group and the non-treated group, demonstrating tumor growth inhibition following FK866 treatment (t-test; p=0.0015). The non-treated group demonstrated an approximately 350% tumor growth increase with a mean tumor volume at the end of experiment (day 32) of 859.21±163.57 mm³, whereas the FK866-treated group demonstrated less than 150% tumor growth increase with a mean tumor volume at the end of experiment of only 300.36±63.67 mm³ (FIG. 15B; p<0.05).

### Example 10

### Sensitivity to NAMPT Inhibitor Correlates with miR-451 Expression Levels in a Xenograft ALL Model

In order to assess a possible association or correlation between miR-451 expression levels and sensitivity to in-vivo treatment of ALL with a NAMPT inhibitor, the effect of the NAMPT inhibitor FK866 was assessed in the presence of elevated levels or lowered levels of miR-451 in ALL xenograft mouse model. First, miR-451 mimic and antagomiR-451 stable NALM-6 cell lines were obtained as described herein and measured for NAMPT expression levels by FACS. A higher expression level of NAMPT was demonstrated in the antagomiR-451 stable line compared to miR-451 mimic stable lines (data not shown), in correlation with the results described in Example 3 herein. The sensitivity of each line to FK866 treatment was examined in vivo in 3 independent experiments, each on a group of 89 mice. Forty-three NSG™ mice injected with NALM-6/antagomiR-451 stable lines were divided into two groups: 18 mice were treated with FK866 (15 mg/kg) once a day for 5 days repeated weekly, and 25 served as controls (treated with saline only). Forty-six mice injected with NALM-6/miR-451 mimic stable lines were divided into two groups: 21 were treated with FK866 (15 mg/kg), and 25 served as controls (treated with saline only). Treatment with the drug started on day 17 after tumor cells injection, when all tumors were clinically evident, and continued once a day for 5 days repeated weekly. Tumors volume was measured daily.

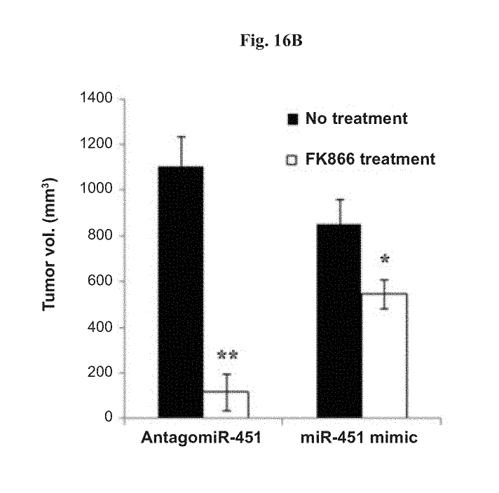

Exemplary tumor growth curves of 30 mice, either treated or not treated with FK866, are depicted in FIG. 16A. Within 9 days of treatment, a significant difference in tumor volume change was observed between NALM-6/antagomiR-451 and NALM-6/miR-451 mimic transduced mice treated with FK866. In addition, a significant difference in tumor growth/volume change was observed between NALM-6/antagomiR-451 mice treated with FK866 as compared to un-treated NALM-6/antagomiR-451 mice (broken blue line vs. continuous blue line in FIG. 16A; p<0.01). Only a moderate difference in tumor growth/volume change was observed between FK866-treated NALM-6/miR-451 mimic mice and untreated NALM-6/miR-451 mimic mice (broken red line vs. continuous red line in FIG. 16A). At the end of experiments, the mean tumor volume of the antagomiR-451 group was 117±81 mm³ in the FK-866-treated mice compared to 1104±134 mm³ in the non-treated group (p=0.000118), while the mean tumor volume in the miR-451 mimic group was 544±64 mm³ in the FK866-treated mice compared to 851±110 mm³ in the non-treated group (p=0.028). As seen in FIG. 16B, the mice xenograft model clearly shows that tumor expressing low levels of miR-451 were significantly more sensitive to FK866 treatment as compared to tumors over expressing miR-451.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for identifying acute lymphoblastic leukemia (ALL) patients most likely to benefit from treatment with a nicotinamide phosphoribosyl transferase (NAMPT) inhibitor, comprising:
obtaining a biological sample from an ALL patient;
determining a level of expression of miR-451 in the biological sample; and
if the level of expression of miR-451 is below a predetermined level, identifying the patient as being likely to benefit from treatment with a NAMPT inhibitor.

2. The method of claim 1, wherein the biological sample is selected from the group consisting of bone marrow, lymph fluid, whole blood, plasma, CNS fluid or serum.

3. The method of claim 1, wherein the subject is an adolescent, child, or infant.

4. The method of claim 1, wherein ALL is pediatric B-cell ALL.

5. A method for prognosis of a treatment modality that inhibits nicotinamide phosphoribosyl transferase (NAMPT) in a subject afflicted with acute lymphoblastic leukemia (ALL), the method comprising:
obtaining a biological sample from the subject;
determining a level of expression of miR-451 in the biological sample; and
if the level of expression of miR-451 is below a predetermined level, correlating the level of expression of miR-451 with a positive prognosis of a treatment modality that inhibits NAMPT.

6. The method of claim 5, wherein the biological sample is selected from the group consisting of bone marrow, lymph fluid, whole blood, plasma, CNS fluid or serum.

7. The method of claim 5, wherein the subject is an adolescent, child, or infant.

8. The method of claim 5, wherein ALL is pediatric B-cell ALL.

9. A method for treatment of acute lymphoblastic leukemia (ALL) in a patient suffering from ALL, comprising:
obtaining a biological sample from the ALL patient;
determining a level of expression of miR-451 in the biological sample; and
if the level of expression of miR-451 is below a predetermined level, treating the patient with a nicotinamide phosphoribosyl transferase (NAMPT) inhibitor.

10. The method of claim 9, wherein NAMPT inhibition is by one or more inhibitors selected from the group consisting of a small molecule inhibitor, antibody, antisense nucleic acid, micro-RNA (miRNA) and RNA interference agent, with the proviso that the inhibitor of NAMPT is not miR-451.

11. The method of claim 9, wherein the NAMPT inhibitor is FK866 or a functional variant thereof.

12. The method of claim 9, wherein the patient is an adolescent, child, or infant.

13. The method of claim 9, wherein the treatment of ALL includes reducing risk of relapse in a patient.

14. A method for early diagnosis of acute lymphoblastic leukemia (ALL) relapse risk in a subject, comprising:
obtaining a biological sample from the subject when first diagnosed as inflicted with ALL;
detecting a level of expression of miR-451; and
diagnosing ALL relapse risk if the level of expression of miR-451 is below a predetermined level.

15. The method of claim 14, wherein the biological sample is selected from the group consisting of bone marrow, lymph fluid, whole blood, plasma, CNS fluid or serum.

16. A method for treatment of pediatric acute lymphoblastic leukemia (ALL) in a subject, comprising:
administering to the subject a therapeutically effective amount of an inhibitor of nicotinamide phosphoribosyltransferase (NAMPT), selected from the group consisting of an antibody, antisense nucleic acid, microRNA (miRNA) and RNA interference agent, thereby treating the patient, with the proviso that the inhibitor of NAMPT is not miR-451.


SEQUENCE LISTING (5 sequences)

SEQ ID NO: 1 - RNA, 21 nt, Homo sapiens
ucgaggagcucacagucuagu

SEQ ID NO: 2 - RNA, 22 nt, Homo sapiens
aaaccguuaccauuacugaguu

SEQ ID NO: 3 - RNA, 17 nt, Homo sapiens
uggauuuuuggaucagg

SEQ ID NO: 4 - RNA, 29 nt, Artificial Sequence
  Sythetic
uggauuuuuggaucaccugauccaaaaau

SEQ ID NO: 5 - RNA, 11 nt, Artificial Sequence
  Sythetic
ucccugaucca